(19) United States
(12) Patent Application Publication
(10) Pub. No.: US 2008/0033431 A1
(43) Pub. Date: Feb. 7, 2008

(54) Position augmenting mechanism

(75) Inventors: Edward K.Y. Jung, Bellevue, WA (US); Eric C. Leuthardt, St. Louis, MO (US); Royce A. Levien, Lexington, MA (US); Robert W. Lord, Seattle, WA (US); Mark A. Malamud, Seattle, WA (US); John D. Rinaldo, Bellevue, WA (US); Lowell L. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the state of Delaware

(21) Appl. No.: 11/478,315

(22) Filed: Jun. 29, 2006

(51) Int. Cl. A61B 17/58; A61B 17/56; A61F 2/30

(52) U.S. Cl. 606/61; 606/60

(57) ABSTRACT

One aspect relates to augmenting positioning of an originally unsecured attachment element with respect to an originally unsecured bridging element. Another aspect relates to a surgical theater mechanism that can be at least partially fabricated at least partially based on position augmenting a first surgical theater element relative to a second surgical theater element. Yet another aspect relates to controlling positioning of a bony element at least partially by augmenting positioning of an originally unsecured attachment element relative to an originally unsecured bridging element. Still another aspect can relate to a shunt tube configured to be augmented secured with respect to a shunt device, at least partially to configure the shunt device into an operable position.

### TECHNICAL FIELD

Certain aspects of this disclosure can relate to, but are not limited to, position augmenting mechanisms and/or techniques.

### BRIEF DESCRIPTION OF THE FIGURES

FIG. 1 is a diagram of a human spine associated with one embodiment of a position augmenting mechanism;

FIG. 2 is an expanded view of the position augmenting mechanism as described with respect to FIG. 1;

FIG. 3, including FIGS. 3a and 3b, are diagrams of another embodiment of the position augmenting mechanism, wherein FIG. 3a shows the position augmenting mechanism in its unassembled state, while in FIG. 3b shows the position augmenting mechanism in its assembled state;

FIG. 4 is a diagram of another embodiment of the position augmenting mechanism that can be configured to be applied to bone fragments of bony elements;

FIG. 5, including FIGS. 5a and 5b, is a diagram of another embodiment of the position augmenting mechanism including a cam;

FIG. 6, including FIGS. 6a and 6b, is a diagram of another embodiment of the position augmenting mechanism including a shape memory (e.g., nitinol-based) fastener;

FIG. 7, including FIGS. 7a and 7b, is a diagram of yet another embodiment of the position augmenting mechanism including a deformable (e.g., crimpable) fastener;

FIG. 8, including FIGS. 8a and 8b, is a diagram of still another embodiment of the position augmenting mechanism including an inflatable (balloon-based) fastener;

FIG. 9, including FIGS. 9a and 9b, is a diagram of still another embodiment of the position augmenting mechanism including a shape memory material;

FIG. 10 is a block diagram of yet another embodiment of the position augmenting mechanism including a processor portion;

FIG. 11 is a diagram of another embodiment of the position augmenting mechanism including an embodiment of an originally unsecured adjustable bridging element;

FIG. 12 is a diagram of one embodiment of the position augmenting mechanism configured as a shunt;

FIG. 13 is a diagram of a portion of the position augmenting mechanism configured as the shunt of FIG. 12;

FIG. 14 is a diagram of one embodiment of the position augmenting mechanism configured as a surgical theater in its assembled state;

FIG. 15 is a diagram of the embodiment of the position augmenting mechanism configured as the surgical theater of FIG. 14 in its un-assembled state;

FIG. 16 is a diagram of another embodiment of the position augmenting mechanism configured as a surgical theater in its expanded or inflated state;

FIG. 17 is a diagram of another embodiment of the position augmenting mechanism;

FIG. 18 (including FIGS. 18a, 18b, and 18c) is a flowchart of an embodiment of positional augmenting, as could be performed by the position augmenting mechanism, as described with respect to FIG. 17;

FIG. 19 is a diagram of another embodiment of the position augmenting mechanism that can be configured to treat bony elements, vertebrae, etc.;

FIG. 20 is a flowchart of an embodiment of positional augmenting, as could be performed by the position augmenting mechanism, as described with respect to FIG. 19;

FIG. 21 is a diagram of another embodiment of the position augmenting mechanism that can be configured as a surgical theater;

FIG. 22 is a flowchart of an embodiment of positional augmenting, as could be performed by the position augmenting mechanism, as described with respect to FIG. 21;

FIG. 23 is a diagram of another embodiment of the position augmenting mechanism that can be configured as a fluid-handling device such as a shunt;

FIG. 24 is a flowchart of an embodiment of positional augmenting, as could be performed by the position augmenting mechanism, as described with respect to FIG. 23;

FIG. 25, including FIGS. 25a and 25b, is a diagram of still another embodiment of the position augmenting mechanism including a positioning and maintaining mechanism;

FIG. 26, including FIGS. 26a and 26b, is a diagram of still another embodiment of the position augmenting mechanism including a positioning and maintaining mechanism; and

FIG. 27, including FIGS. 27a and 27b, is a diagram of still another embodiment of the position augmenting mechanism including a positioning and maintaining mechanism.

### DETAILED DESCRIPTION

At least certain portions of the text of this disclosure (e.g., claims and/or detailed description and/or drawings as set forth herein) can support various different claim groupings and/or various different applications. Although, for sake of convenience and understanding, the detailed description can include section headings that generally track various different concepts associated with claims or general concepts contained therein, and is not intended to limit the scope of the invention as set forth by each particular claim. It is to be understood that support for the various applications or portions thereof thereby can appear throughout the text and/or drawings at one or more locations, irrespective of the section headings.

### 1. Certain Embodiments of a Position Augmenting Mechanism

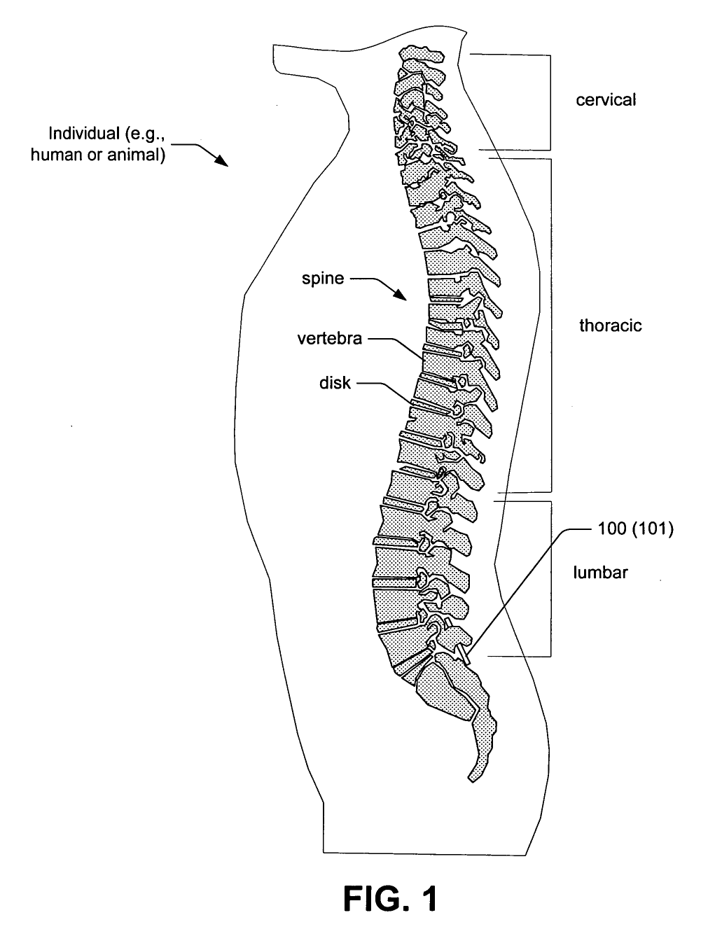

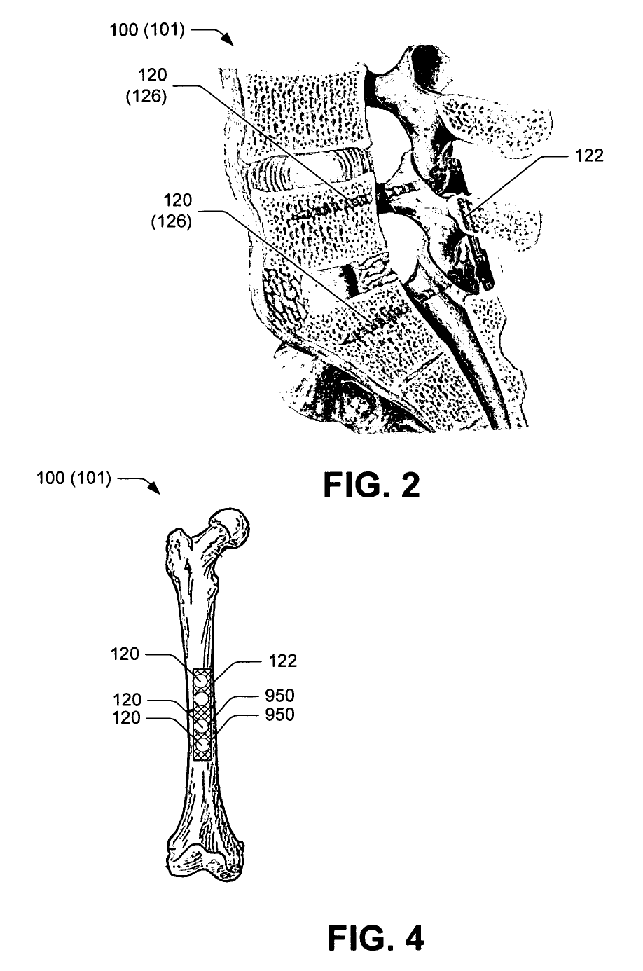

This disclosure describes a number of applications for a variety of embodiments of the position augmenting mechanism 100, as described with respect to FIGS. 1 to 16, for example. Certain embodiments of the position augmenting mechanism may, depending on context for example, utilize a construct. Certain embodiments of constructs as generally known to be used in surgical technologies, etc. Within this disclosure, the term “position augmenting mechanism” 100 can be considered as a mechanism that can include an at least one originally unsecured attachment element 120 and an at least one originally unsecured bridging element 122, as described with respect to FIGS. 2-10.

Within this disclosure, the position augmenting mechanism 100 can, depending on context, include: a) the at least one originally unsecured attachment element 120 being configurable to improve a relative positioning with respect to an at least one originally unsecured bridging element 122 when the former is not in the correct range of desired position(s); and/or b) the at least one originally unsecured attachment element being configurable to maintain a relative positioning with respect to the at least one originally unsecured bridging element when the former is in the correct range of desired position(s). These two aspects of the position augmenting mechanism 100 are intended to be interpreted in the alternative, such that either one can be performed and may satisfy the position augmenting mechanism as described in this disclosure.

Certain embodiments of the position augmenting mechanism 100 can provide a mechanism to enhance healing and/or treatment for an individual having, for example, spinal injuries, sicknesses, problems, etc. By comparison, certain embodiments of the position augmenting mechanism 100 as described with respect to FIG. 4 can be configured to provide a healing mechanism that can be applied to fractured bones, bone elements, etc. Additionally, certain embodiments of the position augmenting mechanism can include shunts, as described in this disclosure. Within this disclosure, the term “individual” can, depending on context, include, but is not limited to: humans or animals.

Certain embodiments of the position augmenting mechanism 100 can be configured to be attached to, maintain, or secure, an item, device, mechanism, bone fragment, boney element, structural component, etc. which can be put in, maintained, repair other elements in, secure other elements in, and/or assembled in a human or animal body. The different embodiments of the position augmenting mechanism 100, as described in this disclosure for example, may be intended to be illustrative in nature, and not limited in scope. For example, the position augmenting mechanism 100 can be applied to, for example, a variety of fractured bones or other boney elements such as a human femur as depicted in FIG. 4, etc. Certain embodiments of the position augmenting mechanism 100 can also be applied to other bones or bony elements, such as, but not limited to, tibias, the fibulas, hand bones, feet bones, bones of the legs, arms, ribs, vertebra(e), etc. Certain embodiments of the position augmenting mechanism 100 that are at least partially attached to one or more bones or bony elements can thereby, depending on context, allow for bone growth, bone fracture repair, spinal fusion, corrective surgery, etc.

While certain embodiments of the position augmenting mechanism 100, as described with respect to FIGS. 1 and 2, is described as being applied to, for example, a set of lumbar vertebra, it is to be understood that similar techniques and mechanisms can also be attached to thoracic or cervical vertebra. Those embodiments of the position augmenting mechanism that are at least partially attached to one or more vertebra may, for example, depending on context, provide for relative fusion of vertebra, (full or partial) removal or repair of disks, spinal “straightening”, spinal repair, etc. While FIGS. 1 and 2 illustrate a pair of vertebrae that can be infused utilizing certain embodiments of the position augmenting mechanism 100, is to be understood that certain embodiments of the position augmenting mechanism 100 can be applied to one or more pairs of vertebrae.

Certain embodiments of the position augmenting mechanism 100, as described in this disclosure, can be applied to a variety of surgery including, but not limited to, surgery of the bones, fractured bones, bony elements, spine, vertebra, etc. Within this disclosure, the term “surgery” can, depending upon context, be intended to be broadly interpreted; and can thereby apply to, but is not limited to, surgeries using relatively large incisions, surgeries using relatively small incisions that may be minimally invasive, surgeries using scopes or other technologies, open surgeries, in-hospital treatments, outpatient treatments, physician office treatments, treatments in the individual's home or other location.

FIG. 1 illustrates one embodiment of the position augmenting mechanism 100, as attached to a human spine including a vertebra. FIG. 2 illustrates an expanded view, as well as the individual components, of the position augmenting mechanism as described with respect to FIG. 1. The embodiment of the position augmenting mechanism 100, as described with respect to FIGS. 1 to 10, can include, but is not limited to, one or more of the originally unsecured attachment element 120 as combined with one or more of the originally unsecured bridging element 122.

Within this disclosure, the term “originally unsecured” when referring to the originally unsecured attachment element 120 and/or the originally unsecured bridging element 122 can indicate, depending upon context, the originally unsecured attachment element 120 being originally unsecured with respect to the originally unsecured bridging element 122 prior to assembly. As such, prior to assembly of the originally unsecured attachment element 120, certain embodiments of the originally unsecured bridging element 122 may not be attached and/or secured with respect to the originally unsecured attachment element 120, and vice versa. As such, the originally unsecured element 120 and/or 122 can be provided with freedom to originally move relatively (not being relatively secured) with respect to each other to allow for relative displacement or positioning prior to and/or during surgery (e.g., non-invasive, open, etc.), etc. During surgery for example, within certain embodiments of the position augmenting mechanism 100, the originally unsecured attachment element 120 may be secured relative to the originally unsecured bridging element 122. Certain embodiments of the position augmenting mechanism 100 can be maintained secured during and/or in some instances following surgery, such as to fuse vertebrae and/or repair broken bones or boney elements, etc.

Within this disclosure, the terms “minimally invasive”, “non-invasive”, “reduced invasiveness”, and/or the like are intended to be used to indicate that such techniques are being applied using reduced-dimensioned incisions, via a body cavity, etc. such as to provide less trauma or damage to the body of the individual, etc. While no surgery can be truly non-invasive, similar techniques such as described herein have greatly reduced injury or trauma to the individual or patient resulting from surgery for a wide variety of surgeries, and have also greatly reduced recovery times. Certain embodiments of the “minimally invasive”, “non-invasive”, and/or “reduced invasive” surgeries, for example, can often utilize scopes of one type of another such that a variety of techniques and/or procedures can be performed. Certain embodiments of the position augmenting mechanism 100 can thereby be applied to either non-invasive and/or open surgery scenarios.

Within this disclosure, the term “maintain” can mean, depending on context, temporarily securing the originally unsecured attachment element 120 relative to the originally unsecured bridging element 122. Such maintaining can be provided, for example, prior to locking the originally unsecured attachment element 120 relative to the originally unsecured bridging element 122 such as by using certain embodiments of the securing or locking mechanism 960 as described in this disclosure with respect to FIGS. 27a and 27b. Other embodiments of the position augmenting mechanism 100 can also utilize the securing or locking mechanism 960 as described in this disclosure. Such maintaining can be performed for a relatively brief duration or a longer duration. Such maintaining can involve relative positioning of the originally unsecured attachment element 120 relative to the originally unsecured bridging element 122 using a variety and range of secured techniques or mechanisms, and certain embodiments of the maintaining can be performed prior to increasing the maintaining, securing, or locking between the elements 120 and 122.

As such, the terms such as “minimally invasive”, “non-invasive”, and/or “reduced invasive” procedures and/or techniques are intended to apply to surgery or other techniques that can result in diminished incision, reduced injury, and/or reduced recovery times as compared with other more traditional surgical techniques and procedures. Often, such “minimally invasive”, “non-invasive”, and/or “reduced invasive” procedures and/or techniques can be performed with less tissue trauma, and therefore can more likely be performed in an outpatient setting or other, instead of using classic in-patient or hospital techniques. Sometimes the minimally invasive procedures can take longer and/or be more technically challenging than conventional “open” procedures. As such, one potentially overriding advantage of minimally invasive procedure may be reduced tissue trauma, reduced incision, reduced recovery duration, etc.

This disclosure describes a number of mechanisms or processes that can be utilized to position, secure, maintain, and/or lock certain embodiments of the originally unsecured bridging element 122 secured with respect to the originally unsecured attachment element 120. Within this disclosure, the positioning, etc. of the originally unsecured bridging element 122 secured with respect to the originally unsecured attachment element 120 can, depending on context, involve moving either element 120 or 122 with respect to the other member, or alternatively both members with respect to each other.

Certain embodiments of the position augmenting mechanism 100 can alternatively be applied to open surgery, in which major incisions may be cut into the individual. Consider, for example, that large constructs (e.g., that might be applied to Scoliosis or other surgeries that may require fusion of multiple vertebral segments), in which one or more of an originally unsecured bridging element 122 such as a rod as described with respect to FIGS. 2, 3a, 3b, 4a, 4b, 5a, 5b, 6a, 6b, 7a, 7b, 8a, 8b, 9a, 9b, 10, and/or 11 can be attached to an originally unsecured attachment element 120 such as a (e.g., pedicle) screw.

Fabrication or assembly of conventional pedicle screws with conventional rods and/or plates can be a complicated two or more person operation. One person can hold, position, maintain, secure, or lock the rod in a suitable position with respect to the pedicle screw using a surgical tool referred to as a rod-holder, while the other person can apply a screw cap such as is known with conventional pedicle screws. As such, assembly of conventional pedicle screws can utilize a considerable amount of hardware as well as medical personnel that might interfere with each other.

Certain embodiments of the position augmenting mechanism 100, as described in this disclosure, therefore can provide a mechanism by which a single medical personnel such as a physician can position, maintain, secure, or lock the originally unsecured attachment element 120 relative to the originally unsecured bridging element 122 such as the rod or plate. In addition, certain embodiments of the position augmenting mechanism 100, as described in this disclosure, can provide a mechanism by which the position augmenting mechanism 100 can maintain and/or lock the originally unsecured attachment element 120 relative to the originally unsecured bridging element 122 such as the rod or plate. Within this disclosure, such terms as “maintain and/or lock” can be evanescent (e.g., temporarily or easily reversible) or relatively permanent.

Certain embodiments of the originally unsecured attachment elements 120 can include but is not limited to, depending upon context: assemblies that can include but are not limited to: screws, pins, bolts, fasteners, clamps, crimping mechanisms, etc. For example, the originally unsecured attachment elements 120 as described with respect to FIG. 2 can include, but is not limited to, one or more fasteners 126 (e.g., a screw, such as a pedicle screw, such as can be used in constructs for spinal surgery, etc.) which are intended to be illustrative, but not limiting in scope. For example, a variety of types of attachment elements or fasteners may be used while remaining within the scope of the present disclosure that may be attached to bones, bone fragments, bony elements, and/or other structural members (even to create a shunt, a surgical theater, or other constructs as described by certain embodiments of this disclosure).

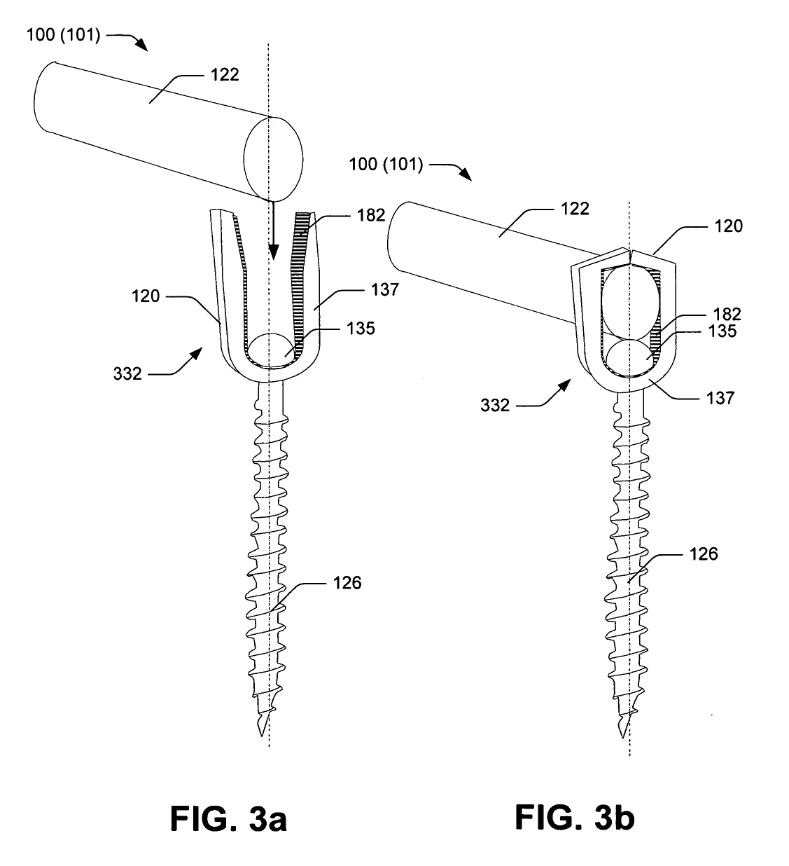

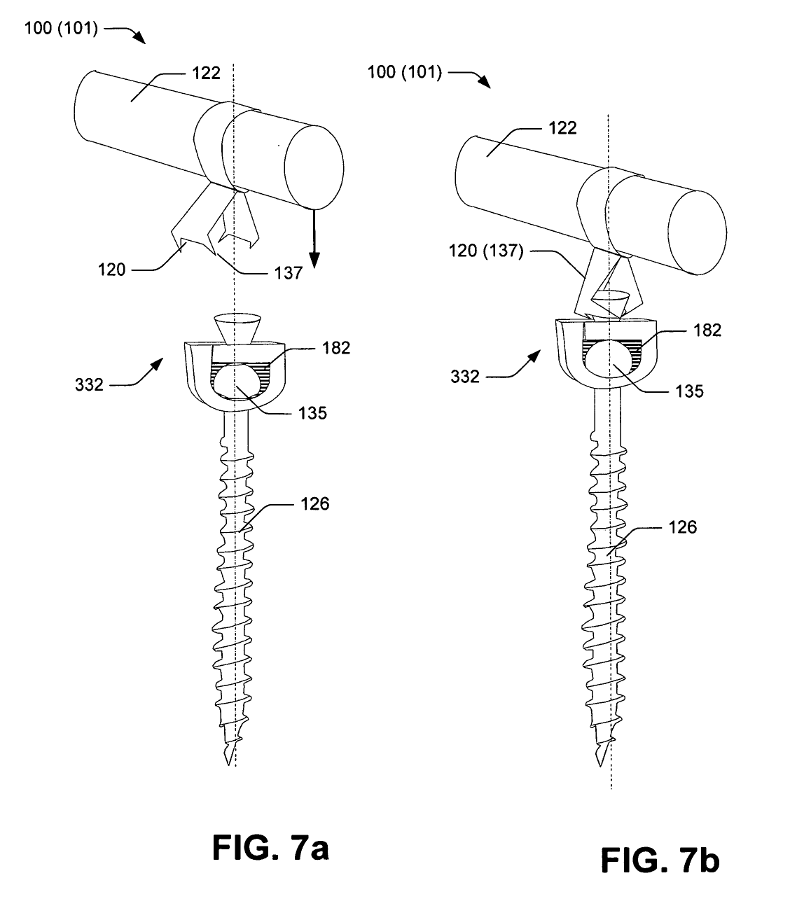

FIGS. 3a, 3b, 5a, 5b, 6a, 6b, 7a. 7b. 8a. 8b. 9a. and 9b show a variety of embodiments of an attachment-enhancing member 182 which can be configured to improve the attachment of the originally unsecured attachment elements 120 with respect to the originally unsecured bridging element 122, or vice versa. As described with respect to FIGS. 7a and 7b, for example, certain embodiments of the attachment enhancing member 182 can e provided as a portion of the originally unsecured bridging element 122, which may be physically or operationally separated from the originally unsecured attachment elements 120, or vie versa. In certain embodiments, the attachment-enhancing member 182 can be configured to even unevenness of the contact area, and/or increase the attachment area between the originally unsecured attachment elements 120 with respect to the originally unsecured bridging element 122.

Certain embodiments of the attachment-enhancing member 182 can be configured as a gasket or sealing member that can reduce relative slippage between the inter-fitting elements. Certain embodiments of the attachment-enhancing member 182 can be applied as a liquid, a solid, or even a gas, and may even change state as the position augmenting mechanism 100 approaches in its final (assembled) position or state. Certain embodiments of the attachment-enhancing member 182 can include an elastomeric, rubber, or other deformable material that can deform away from pressure points and into spaces, etc. to increase the contact area between the originally unsecured attachment elements 120 with respect to the originally unsecured bridging element 122.

Another embodiment of the attachment-enhancing member 182 (which in certain embodiments can attached to the originally unsecured attachment element 120 and/or the originally unsecured bridging element 122) can be configured to closely conform to the contour, structure, and/or shape of the originally unsecured bridging element 122. This close conformation can be intended to increase the fastening strength between the originally unsecured attachment element 120 and the originally unsecured bridging element 122, in many embodiments by increasing the contact surface area there between when in the assembled position. These embodiments of the attachment-enhancing member 182 may not need the resilience or elasticity as compared to those embodiments of the attachment-enhancing member described elsewhere which can deform upon attachment, but instead may be pre-formed or molded into a suitable shape or configuration to conform between the appropriate portions of the originally unsecured attachment element 120 and/or the originally unsecured bridging element 122.

While a number of embodiments and materials of the attachment enhancing member 182 are described in certain embodiments within this disclosure (which can include but are not limited to certain embodiments of the deformable attachment-enhancing members and/or the “pre-formed” attachment members), it is to be understood that other configurations, materials, or mechanisms can be utilized as certain embodiments of the attachment-enhancing member 182 such as would be known to those skilled in the fastener technologies, etc. Certain embodiments of the position augmenting mechanism 100 can be fabricated with no attachment enhancing member 182 between the originally unsecured attachment element 120 and the originally unsecured bridging element 122, for example.

While this disclosure describes a number of embodiments of the position augmenting mechanism 100 by which the originally unsecured attachment element 120 attaches and/or de-attaches from the originally unsecured bridging element 122, it is also envisioned that there may be certain embodiments of the position augmenting mechanism in which the originally unsecured bridging element 122 can attach to, and/or de-attaches from certain embodiments of the originally unsecured attachment element 120. Such modifications of and/or interchanging of operations or functionalities of certain elements of the position augmenting mechanism 100 are intended to be within the scope of the present disclosure.

Certain embodiments of the one or more originally unsecured bridging element 122, as described in this disclosure, can include depending upon context, but is not limited to: rods, plates, connecting members, bridging members, etc. by which one or more members are spaced, positioned, locked, maintained, secured, etc. with respect to another one or more member. In certain embodiments, the members being spaced can include, but are not limited to, bones, bone fragments, bony elements, vertebra, assembly structures, mechanical components, etc. The shapes and/or configurations of the elements 120 and 122 within this disclosure is intended to be illustrative in nature, but not limiting in scope.

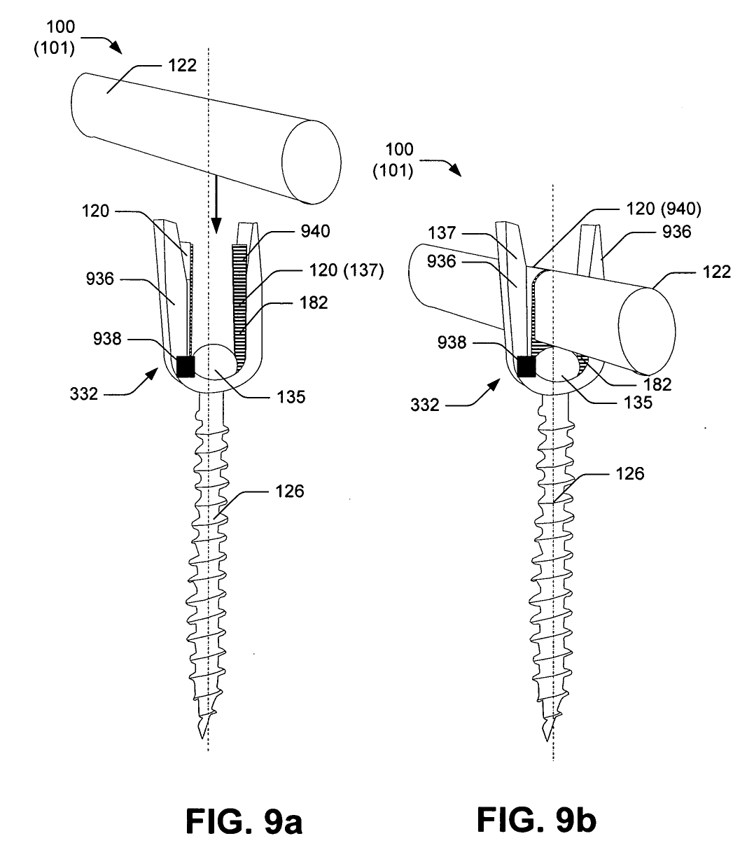

Certain embodiments of the position augmenting mechanism 100, as described in this disclosure, can be assembled and/or attached at least partially based on recognition of and/or as a result of a context. Certain embodiments of the context can indicate and/or be used to display or determine, for example, some condition or parameter (or absence thereof), etc. that the presence of can indicate that the position augmenting mechanism should be used, utilized, attached, applied, fabricated, constructed, etc. Certain embodiments of the context can include, but is limited to, a position context, a pressure context, a proximity context, or any other context by which the position augmenting mechanism 100 can be configured to be attached and/or assembled. Certain embodiments of the assembling and/or attaching of the position augmenting mechanism 100 can be at least partially manual (a surgeon pressing two parts together), or at least partially automatically applied. Certain embodiments of the position augmenting mechanism 100 can include a sensor (not shown) that, upon some aspects such as relative positioning, securing, maintaining, locking, etc. of the elements 120 and 122, can determine relative position of the elements 120 and 122, and/or relatively secure the elements. There may be a variety of sensors that may be utilized including, but not limited to: contact sensors, proximity sensors, sensors that allow electric current to flow between members upon contact there between (e.g., as described in this disclosure with respect to a context sensor 938 as described with respect to FIGS. 9a and 9b), etc. Within this disclosure, a context sensor can mean, depending on usage, a mechanism that can be used with certain embodiments of the position augmenting mechanism 100 to ensure that the position augmenting mechanism is in a state or position such that it can readily be assembled, actuated, etc.

Certain embodiments of the position augmenting mechanism can be configurable to, based at least in part on a context, secure an originally unsecured bridging element 122 relative to an originally unsecured attachment element certain embodiments of the position augmenting mechanism can utilize originally unsecured attachment element, which may also include, e.g., a cup that can be associated with certain embodiments of the fastener 126 such as the pedicle screw. In certain embodiments, prior to attachment, the position augmenting mechanism can move originally relative to the originally unsecured attachment element. Certain embodiments of the one or more originally unsecured attachment element 120 can include, but is not limited to, the originally unsecured attachment element that can be applied, for example, to a bone, to a part of a bone (e.g., bone fragments, bony elements, a portion of a vertebrae, etc.) using threads or other connective mechanisms.

Within this disclosure, certain embodiments of the originally unsecured attachment element can include, but is not limited to, an attachment mechanism having threads that can secure into a bone member (e.g., the bone member can include but is not limited to, cancellous or spongy bone, or such bones as vertebral bodies (spine), long bones (femur), hand bones, foot bones, etc.). In effect, there might be relatively few bones in which a user, such as a physician, cannot insert some modified embodiments of the originally unsecured attachment element of one configuration or other. A pedicle screw is an example of one embodiment of the originally unsecured attachment element.

Within this disclosure, the terms “fastener”, “screw”, “bolt”, or “pin” can be used interchangeably as to include, but not be limited to, a threaded or non-threaded axially extending member. In certain embodiments, friction can apply to between the pin and it's mating surface to exert pressure between the pin against bone to, in effect, hold the pin in place. As such, fasteners, screws, bolts, and/or pins can be applied to a variety of situations as described in this disclosure. In certain embodiments, fasteners such as pins can be at least partially threaded such as to extend through at least a portion of a bone while not another, such as is known for traction. In certain embodiments, fasteners such as screws can use to relatively connect both fragments or bony elements (while allowing little relative or limited relative motion) such as in fusing spinal vertebrae or connecting broken or fractured bone ends.

By allowing certain embodiments of the position augmenting mechanism 100 to be applied through a surgical incision, the time of the surgical techniques utilizing certain embodiments of the position augmenting mechanism 100 can be considerably reduced. As such, certain embodiments of position augmenting mechanism 100 can thereby be utilized to realize increased efficiency for spinal surgery, bone fracture surgery, etc. This can also allow for increased ease of certain minimally invasive surgical approaches and/or devices. For instance, certain embodiments of the originally unsecured attachment element 120 and the originally unsecured bridging element 122, as described with respect to FIGS. 2, 3a, and/or 3b, 4, 5a, 5b, 6a, 6b, 7a, 7b, 8a, 8b, 9a, and/or 9b can be each inserted into the individual through relatively small relative incision for each element, and then all of the elements can be assembled in its final state therein. In standard surgical scenarios an incision is made in the back (either large for open procedures or small for minimally invasive approaches) and the boney elements of the spine are exposed and instrumented. Currently once a screw is placed in the spine, to bridge that screw to another screw in the spine requires the attachment of a rod or plate between the screws. Once the bridging element is in proximity to the screw, it can be definitively secured through the attachment of a securing nut or by tightening a bolt adjacent to the screw. Certain embodiments of the position augmenting mechanism 100 may allow for a more efficient and easy attachment of the bridging element by reducing the steps requisite to achieve definitive fastening (e.g., by limiting the securing nut).

Certain embodiments of the originally unsecured attachment element can also be used to apply relative axial pressure (e.g., either pull together axially, or separate axially—not illustrated) between two bony elements or portions, such as ends of fractured bones, together to be substantially axially approximated. In certain embodiments, the ends of the fractured bones can be situated in proximity to one another. Consider that though alignment of bony parts is intended be included within this disclosure, precise alignment may not be possible or practical due to forces that may be applied to one or more of the bony parts due to interaction between the bone parts and muscles, ligaments, tendons, etc.

A number of illustrative embodiments of the position augmenting mechanism 100 is now described in this disclosure. FIG. 1 shows one embodiment of the position augmenting mechanism 100, that may not be actuated (e.g., be configured, be displaced, be controlled, be fired, etc.) such as to be assembled, etc., until a context indicates the position augmenting mechanism should be actuated and/or assembled. In certain embodiments, as described in this disclosure with respect to FIGS. 2 and/or 4, for example, the position augmenting mechanism 100 can include one or more originally unsecured attachment element 120 as well as one or more originally unsecured bridging element 122, which can be fastened relatively easily to simplify installation and can be installed using minimal invasive techniques. In certain embodiments, as described in this disclosure, the position augmenting mechanism 100 can also be “reversed assembled” or “de-assembled”, such as to provide relatively-simplified removal of the one or more originally unsecured attachment element 120 and/or the one or more originally unsecured bridging element 122, such as when no longer necessary or to allow the position augmenting mechanism 100 to be removed and/or repaired. Certain embodiments of such originally unsecured attachment elements could, for example, be applied during surgery (e.g., non-invasive, open, etc.), such as orthopedic surgery. As such, the reversibility may also be desirable in orthopedic surgery applications such as if it may be desired to remove a pin or plate as well as the associated fasteners.

Certain embodiments of the position augmenting mechanism 100, as described with respect to FIG. 1, can also be considered as and/or include a construct 101. In certain embodiments, as described in this disclosure, the construct 101 can thereby include but is not limited to one or more originally unsecured attachment element(s) 120 plus one or more originally unsecured bridging element 122. Certain embodiments of the construct can include, but is not limited to, some mechanism which can be constructed within a body of an individual such as during surgery (e.g., non-invasive, open, etc.).

There can be a variety of motion(s) that can be provided, desired, and/or allowed between the originally unsecured attachment element 120 and/or the one or more originally unsecured bridging element 122. For instance with a variety of surgeries or repair involving relative spinal vertebra, it may be desired to substantially anchor or fuse the relative spinal vertebrae along all three axes (spinal axial and two orthogonal spinal lateral), such that the relative spinal vertebrae can thereupon be considered as a single fused structure. Consider that it might be desired to fuse vertebrae to provide cervical, thoracic, and/or lumbar spinal support for certain individuals. Individuals having such surgery may, for example, experience reduced pain and injury associated with further spinal vertebra injury. In certain spinal injury or illnesses, such as Scoliosis or other spinal deformation, a number of spinal vertebrae may be relatively anchored or fused during surgery to limit relative motion along one, two, or three spinal axes. Such fusion can be performed incrementally quickly, and can be intended to allow “reshaping” or “straightening” of the spine in a manner that is well known and/or understood by those skilled in the art, such as spinal surgeons. This disclosure describes a number of embodiments of techniques and/or devices by which such constructs and/or position augmenting mechanisms 100 can be relatively simply applied, removed, and/or adjusted both during and following surgery.

Certain embodiments of the originally unsecured attachment element 120 and the one or more of an originally unsecured bridging element 122 can be configured to provide motion along one or more of the orthogonal axis. For example, to promote bone growth, it may be desired to allow some relative motion between the bone ends or the bony elements following a broken or fractured bone(s), during dental treatments such as orthodontia, etc. In certain instances, for example, relative motion can be allowed in certain embodiments of the position augmenting mechanism 100 between the bone ends or the bony elements may be necessary for the body to generate “growth signals” from which the mechanism of new bone growth can be enhanced or provided. The amount of relative motion of the bone ends or the bony elements should be within certain prescribed limits that can vary based, for example, on such factors as the individual's age and condition, the type or severity of the injury or illness, whether the individual is healthy, conscious, bed-ridden, youthful, the particular bone that is fracture broken, the particular type of treatment and expected type of recovery, the particular condition or configuration of the bone ends or the bony elements, etc. For example, if there is a relatively large amount of relative motion between the bone ends or the bony elements, then the motion will limit the bone repair. If there is relatively little motion between the bone ends or the bony elements, then the bone-growth signals to generate the new bone growth could be limited. As such, certain embodiments of the position augmenting mechanism 100 can be applied to be suited to particular individual, illness, and/or injury based at least in part on the knowledge, skill, and/or techniques of the practitioner, such as a surgeon. Based on such parameters as these, surgeons such as spinal surgeons, etc. can select a suitable embodiment (size, strength, relative motion) of the position augmenting mechanism 100 to allow for suitable injury or illness repair.

There can be a variety of techniques and/or utilized mechanisms, as described in this disclosure, by which various embodiments of the position augmenting mechanism 100 can be assembled, secured, maintained, locked, etc. which can vary from largely manual assembly (e.g., manually pressing the elements 120 and 122 together relatively) to largely automated assembly (being actuated utilizing a microprocessor, processor, computer, etc.). This disclosure describes a number of manual techniques, but further components such as processor-based devices, computer-based devices, and/or controller-based devices may be utilized to accomplish automated assembly, further components may be utilized. Certain embodiments of the position augmenting mechanism 100 can thereupon also include, but is not limited to, the context sensor 938 and one or more assembling mechanism (not shown) which can be utilized to assemble the position augmenting mechanism 100. Certain embodiments of the context sensor 938 may be configured to determine the occurrence, or absence of, the context that may indicate the position augmenting mechanism 100 should be actuated. For example, certain embodiments of the context sensor may utilize such context detects as the proximity detector, position detector, or other context detector which may or may not utilize computer or controller technology as described in this disclosure. Certain embodiments of a proximity-context sensor may, for example, sense a suitable proximity of the one or more originally unsecured attachment element 120 and/or the one or more originally unsecured bridging element 122 which can thereupon be assembled into the position augmenting mechanism 100.

Upon indication by the context sensor that certain embodiments of the position augmenting mechanism 100 can be assembled, certain embodiments of the context sensor 938 can thereupon actuate the one or more assembling mechanism to thereby provide for assembly of certain embodiments of the position augmenting mechanism and achieve a certain level of automation of assembly as described in this disclosure.

Within this disclosure, certain embodiments of the context sensor may also be processor based. For example, with certain embodiments of the position augmenting mechanism 100, a practitioner such as surgeon can sense when one or more originally unsecured attachment element 120 and/or the one or more originally unsecured bridging element 122 are relatively positioned with respect to one another to permit assembly thereof. Thereupon, the practitioner can apply for suitable force, actuation, and/or relative positioning, securing, maintaining, locking, etc. of the one or more originally unsecured attachment element 120 and/or the one or more originally unsecured bridging element 122 to provide assembly of certain embodiments of the position augmenting mechanism 100.

FIG. 3a illustrates an embodiment of the position augmenting mechanism 100 including a relatively unassembled embodiment of the one more originally unsecured attachment element 120 relative to a soon-to-be assembled one or more originally unsecured bridging element 122. FIG. 3b illustrates an embodiment of the position augmenting mechanism 100 including a relatively assembled embodiment of the one more originally unsecured attachment element 120 relative to the one or more originally unsecured bridging element 122. Certain embodiments of the originally unsecured attachment element 120 thereby can include, but are not limited to, a fastener portion (not shown), a context-based assembly portion 332, and/or a relative connection portion (not shown). Within this disclosure, certain embodiments of the fastener portion (not shown) can, depending on context, act to secure the position augmenting mechanism 100 in its attached, maintained, or secured position such as to a bone, a bone fragment or a bony element, and/or to a structural component such as a shunt, a surgical theater, and/or a traction device, and or other devices or applications as described in this disclosure.

Within this disclosure, certain embodiments of the context-based assembly portion 332 can, depending on context, be configured to provide for assembly between the originally unsecured attachment element 120 relative to the one or more originally unsecured bridging element 122 to assemble certain embodiments of the position augmenting mechanism 100. Additionally, certain embodiments of the context-based assembly portion 332 can, depending on context, be configured to provide for disassembly between the originally unsecured attachment element 120 relative to the one or more originally unsecured bridging element 122 to disassemble and/or remove certain embodiments of the position augmenting mechanism 100. Within this disclosure, certain embodiments of the context-based assembly portion 332 can provide a connection and/or motion between one or more of the originally unsecured attachment element 120 relative to one or more of the one or more originally unsecured bridging element 122, as desired such as to provide the desired functionality or operation of the position augmenting mechanism 100. For instance, certain embodiments of the originally unsecured attachment element 120 may be configured such as to have their axial direction angled at some unusual angle relative to the originally unsecured bridging element 122 (e.g., non-orthogonal) such as illustrated with respect to FIG. 2. For example, an angle between the one or more originally unsecured attachment element 120 and/or the originally unsecured bridging element 122 may be selected-based, at least in part, on how to best secure the originally unsecured attachment element to the spine, vertebra, bone fragment, bony element, structural element such as a portion of a surgical theater, etc. such as described in this disclosure.

Certain embodiments of the fastener 126 can be configured in the position augmenting mechanism 100 in its originally unsecured state, as described with respect to FIGS. 3a, 5a, 6a, 7a, 8a, and/or 9a, for example, may include a head portion 135, such as a rounded head for a pedicle screw. The fastener 126 can be sized to extend through an aperture (not shown) formed in a positioning portion 137 of the originally unsecured attachment element 120 such as to provide some play between the fastener 126 and the positioning portion 137 when the position augmenting mechanism is in its originally unsecured state. By comparison, the head portion 135 can be sized to not pass through the aperture formed in the positioning portion. As such, when certain embodiments of the originally unsecured attachment element 120 is in its originally unsecured state, there can be some angular play between the originally unsecured attachment element 120 and the originally unsecured bridging element 122, such as to allow attachment of the fasteners at a variety of angles as described with respect to FIG. 2.

By comparison, the fastener 126 can be configured in the position augmenting mechanism 100 in its secured state such as by securing of the originally unsecured attachment element 120 with respect to the originally unsecured bridging element 122, as described with respect to FIGS. 1, 2, 3b, 5b, 6b, 7b, 8b, and/or 9b. During securing of the originally unsecured attachment element 120, pressure can be exerted between the positioning portion 137 and/or other portion of the originally unsecured attachment element 120 and the head portion (e.g., rounded) 135 of the fastener 126. Therefore, with certain embodiments of the position augmenting mechanism 100, securing the unsecured bridging element 122 within the originally unsecured attachment element 120 may have the effect of securely positioning the unsecured bridging element 122 with respect to the originally unsecured attachment element 120, as well as solidifying the various portions of the originally unsecured attachment element 120 in a desired range of positions as well as a desired range of angles.

Certain embodiments of the originally unsecured attachment element 120 can be configured to maintain its securement and positioning relative to, the unsecured bridging element 122. For instance, appropriate mating elements within elements 120 and/or 122 can be grooved, etched, coated, roughened, and/or otherwise configured as to reduce or limit motion between relative securing elements or portions thereof.

As described with respect to this disclosure, certain embodiments of the at least one originally unsecured attachment element 120 of certain embodiments of the position augmenting mechanism 100 may be configured to be “primarily-manual” such as to be configured to provide for relatively quick assembly, attachment, and/or removal of one or more component within the position augmenting mechanism 100. A variety of embodiments of the at least one originally unsecured attachment element can thereby be configured to relatively secure the originally unsecured bridging element 122 in a desired fashion and/or utilizing a particular technique, certain embodiments of the at least one originally unsecured bridging element 122.

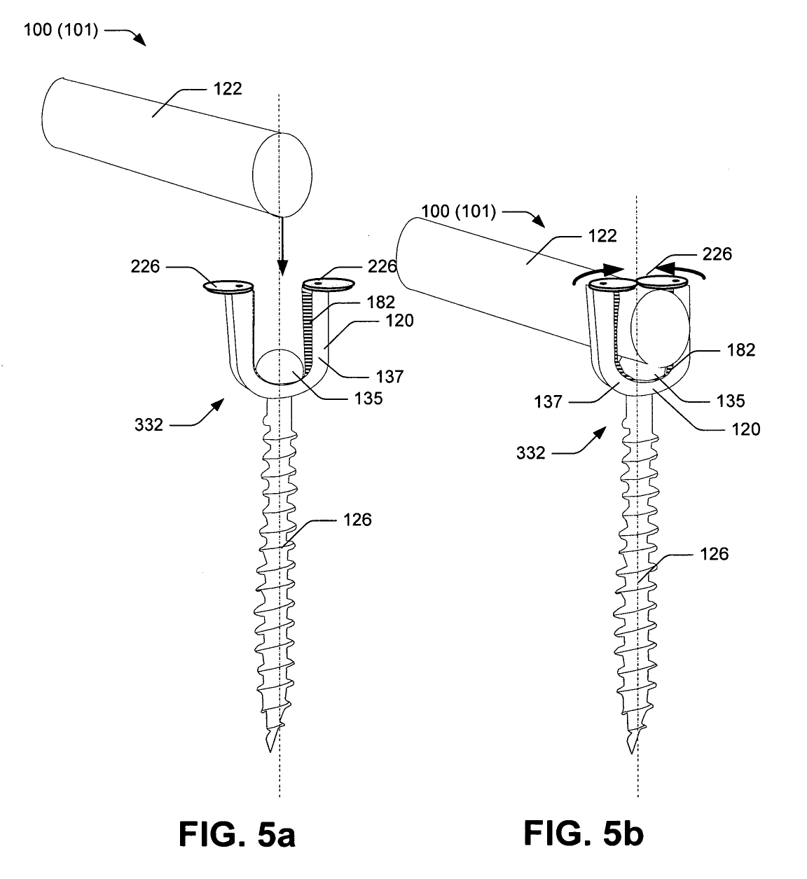

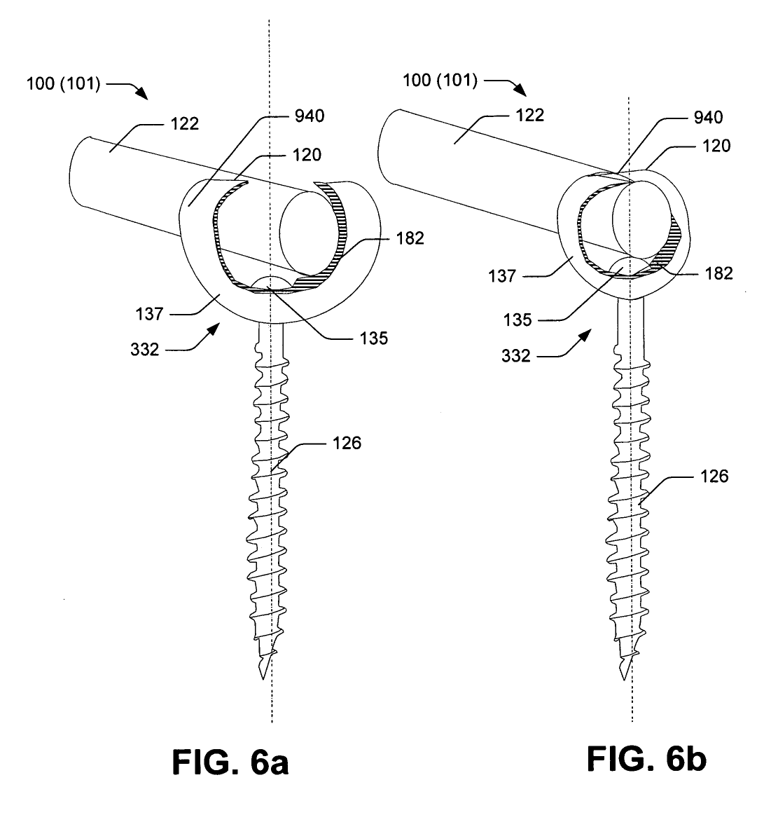

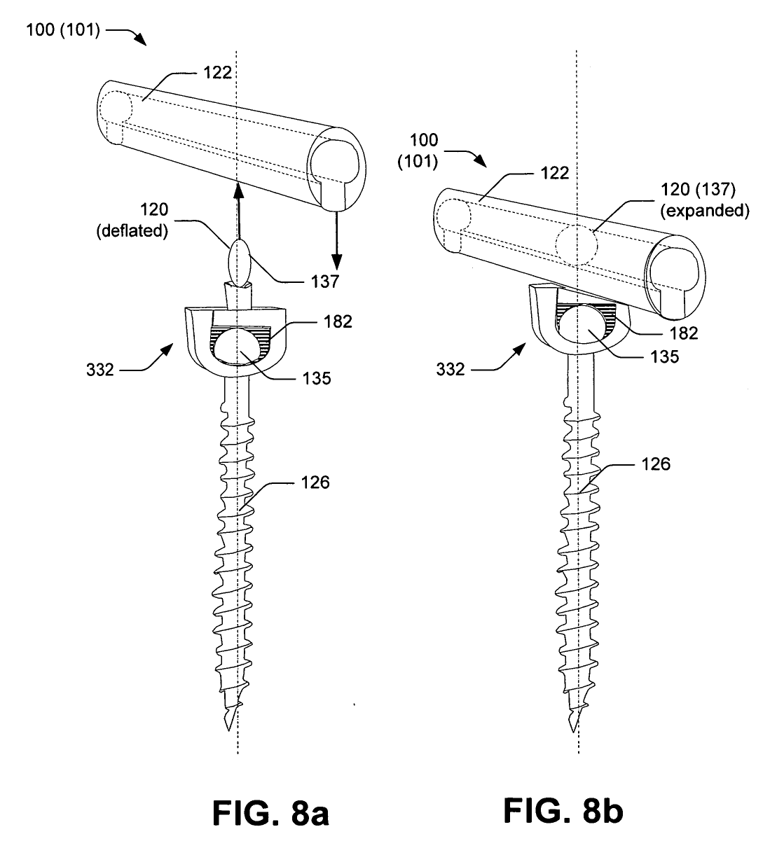

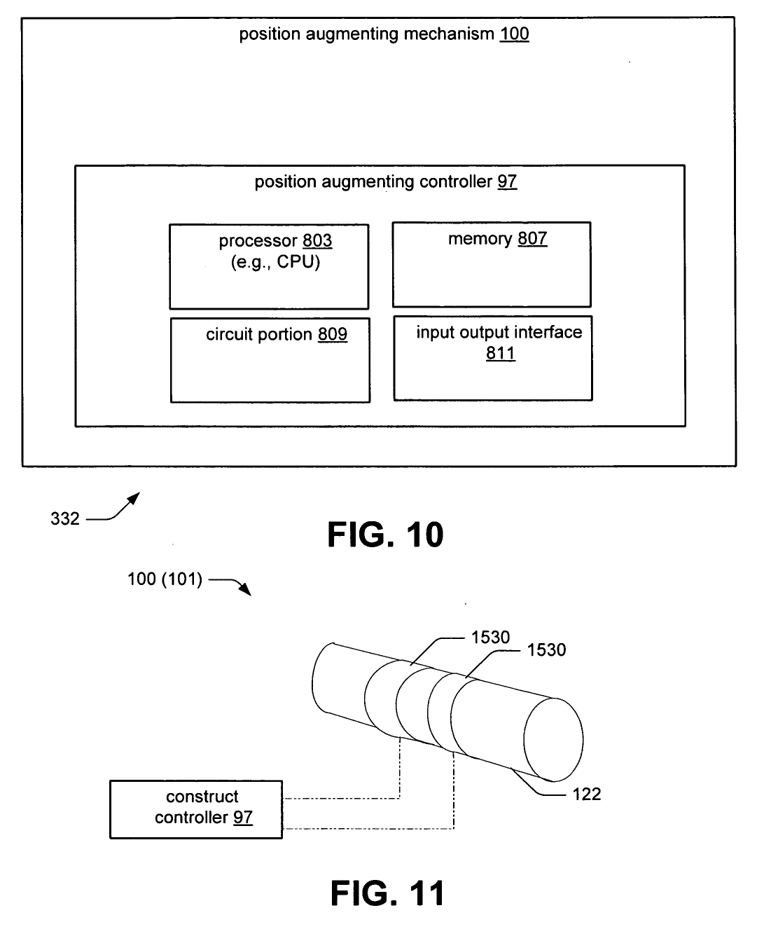

Certain embodiments of the at the least one originally unsecured attachment element can be configured, as described with respect to FIGS. 3a and 3b, to include certain embodiments of an at least one positioning portion 137 that when configured as illustrated in FIG. 3b, can secure the originally unsecured bridging element 122 with respect to the originally unsecured attachment element 120. There are a variety of embodiments of the at least one positioning portion 137 that can include, but is not limited to, deformable members as described with respect to FIGS. 3a and 3b, a rotational cam as described with respect to FIGS. 5a and 5b, a shape memory (e.g., nitinol) fastener as described with respect to FIG. 6, a crimpable portion as described with respect to FIG. 7, an inflatable (e.g., balloon) attachment portion as described with respect to FIG. 8, and/or a processor-based portion as described with respect to FIG. 10. Consider that these embodiments of the at the least one originally unsecured attachment element of the position augmenting mechanism 100 can thereby utilize a variety of technologies such as, but not being limited to, being one or more of crimp-based, cam based, microprocessor based, spring based, etc. such as described in various portions of this disclosure.

Certain implementations at least one originally unsecured attachment element might include a one or more cams 226, as described with respect to FIGS. 5a and/or 5b that can be rotated to an unsecuring position, as described with respect to FIGS. 5a to permit insertion of the originally unsecured bridging element 122; and thereupon the one or more cams 226 can be rotated to secure the one or more originally unsecured bridging element 122 with respect to the one or more originally unsecured attachment element 120. For example, certain embodiments of the one or more cams 226 can be integrated on the one or more originally unsecured bridging element 122 and/or the one or more originally unsecured attachment element 120 as illustrated in FIG. 5a; and upon rotation as described with respect to FIG. 5b, the cam can apply suitable pressure between the originally unsecured bridging element 122 and the originally unsecured attachment element 120 to maintain the originally unsecured attachment element 120 and/or the originally unsecured bridging element 122 in place, such that they would not likely be relatively dislodged unless acted upon by external tool, or a double walled screw. With certain embodiments, one or more of the cam surfaces may be angled such that when the one or more cams are rotated such as to be biased against, e.g., the one or more originally unsecured bridging element 122, the securing of the one or more originally unsecured bridging element 122 can be enhanced. In another embodiment, not illustrated, a quick-lock assembly which may be similar in operation to a variety of embodiments of quick-lock devices such as, but not limited to, a so called “molly bolt” (as are known as often being used and are generally known in the fastener arts to secure pictures, etc. to drywall, for example) can be provided to secure the one or more originally unsecured bridging element 122 with respect to the one or more originally unsecured attachment element 120. While the embodiment of cams 226 as described with respect to FIGS. 5a and 5b are illustrated as rotating in a substantially horizontal plane, as referenced to the FIGS., any suitable cam rotation and/or configuration is intended to be within the intended scope of the present disclosure.

Another embodiment of the position augmenting mechanism 100 as described with respect to FIGS. 3a, 3b, 7a, and/or 7b may include variations of a deformable and/or a crimpable attachment member. Certain embodiments of crimping are generally understood, and may involve bending relatively deformable elements such as to secure the one or more originally unsecured bridging element 122 with respect to the one or more originally unsecured attachment element 120. In certain embodiments, the deformable portion may include an expansive version such as a miniaturized balloon that can interfit within a groove, for example, formed in the one or more originally unsecured bridging element 122. In certain embodiments, the deforming attachment members can thereby be deformed elasticity such as with an expansive balloon in fluid communication with a portion. Certain embodiments of the deforming attachment members can thereby be inflatable such as to maintain relative positioning within, e.g., a groove or lock such as described with respect to FIGS. 8a and 8b, such as with a balloon portion 832 can be configured to be expanded to maintain the originally unsecured attachment element 120 and the originally unsecured bridging element 122 relatively inter-positioned, inter-locked, inter-secured, inter-maintained, etc.

As such, the position augmenting mechanism can be fixed in place so long as the balloon portion is inflated. Deflating the inflatable portion or balloon portion when the position augmenting mechanism 100 is in its secured state, as described with respect to FIGS. 8a and 8b, can conversely permit the originally unsecured attachment element 120 and the originally unsecured bridging element 122 to be de-assembled such as to provide for removal of certain embodiments of the position augmenting mechanism 100. In certain implementations, expanding of the balloon or expansible portion can be achieved, e.g., by controlling an applied gas reservoir (not illustrated) that can supply a gas or liquid into the balloon, and in certain embodiments, the applied gas reservoir can be routed and/or valved utilizing a suitable mechanism. Within this disclosure, the term “balloon” is intended to describe an inflatable mechanism which can fit in a groove, slot, etc., which when inflated can maintain its associated element 120 or 122 in contact with the respectively connecting element 122 or 120. In certain embodiments, the expansible portion as described with respect to FIGS. 8a and/or 8b can also include, but is not limited to, a micro electro-mechanical system (MEMS) actuator device such as described with respect to the inflatable embodiment of the at the least one originally unsecured attachment element. Certain implementations of the position augmenting mechanism 100 might utilize imaging and/or sensing techniques wherein the user can determine, and the position augmenting mechanism can be actuated in a variety of techniques, as described in this disclosure.

FIG. 9, including FIGS. 9a and 9b, illustrates another embodiment (from FIGS. 6a and 6b) of the position augmenting mechanism 100 that can include, but is not limited to, the one or more originally unsecured attachment element 120 (which may include one or more shape memory elements 940), the one or more originally unsecured bridging element 122, a sleeve 936, and the context sensor 938. In certain embodiments, the context sensor 938 may utilize a variety of power sources such as, e.g., either battery, MEMS, wireless-transmitted power, or other suitable power technologies.

Within this disclosure, the term “shape memory”, “shape memory alloy”, or similar shape memory related term can mean, depending on context, but is not limited to, being biased into a secondary position prior to assembly or actuation, which when actuated such as by application of an electric current, can return to its original (non-biased) position. In one embodiment, the shape memory elements 940 may be maintained within a sleeve such as to be protected thereby during insertion, assembly, operation, removal, or other operation. Considering the embodiments of the shape memory elements 940 as described with respect to FIGS. 6a, 6b, 9a, and 9b, though not numbered in 6a and 6b, a great number of modifications of the shape memory element material(s), configuration(s), and/or design(s) can be provided.

Following the insertion of the one or more originally unsecured bridging element 122 relative to the one or more originally unsecured attachment element 120, the one or more originally unsecured attachment element 120 can thereupon be deformed to attach to or secure to the one or more originally unsecured bridging element 122. Thereupon, the shape of the one or more originally unsecured attachment element 120 can be actuated to deform to, in one embodiment, maintain and/or secure the one or more originally unsecured bridging element 122. Such locking can be affected, for example, by actuating the shape memory elements 940 into their original “un-deformed” state such as to secure the one or more originally unsecured bridging element 122 in position. In certain embodiments, the sleeve 936 can be configured to protect the one or more originally unsecured attachment element 120 and/or the one or more originally unsecured bridging element 122 such as are contained therein.

In certain embodiments of the of the position augmenting mechanism 100, such as described with respect to FIGS. 9a and 9b, the securing of the one or more originally unsecured bridging element 122 can be substantially automated as a result of the insertion of the one or more originally unsecured bridging element 122 into the one or more originally unsecured attachment element 120. For instance, the insertion of certain embodiments of the one or more originally unsecured bridging element 122 into the one or more originally unsecured attachment element 120 can cause the one or more shape memory elements 940 into its original state, such as may be designed to secure the one or more originally unsecured bridging element 122 in its secured state.

In certain embodiments, for example, positioning the one or more originally unsecured bridging element 122 can be configured to establish an electric current through the one or more originally unsecured attachment element 120 from the context sensor 938. For example, a closed loop circuit can be temporarily or permanently established including the context sensor 938, the one or more originally unsecured bridging element 122, and/or the one or more originally unsecured attachment element 120 in a variety of configurations such as would be understood to those skilled in the electronics circuitry art. By applying the electricity from the power source to certain embodiments of the one or more originally unsecured attachment element 120 including the one or more shape memory elements 940, the one or more shape memory elements 940 can be un-deformed upon actuation into their original shapes (the shapes they were in prior to deformation). Upon actuation, certain embodiments of the unsecured bridging element 122 could be suited to maintain the one or more originally unsecured bridging element 122 in its secured or assembled position. In certain embodiments, a proximity sensor or other suitable circuit can also be provided to detect relative positioning of the one or more originally unsecured bridging element 122 and the one or more originally unsecured attachment element 120.

Within this disclosure, the one or more originally unsecured bridging element 122 as described with respect to the position augmenting mechanism 100 and/or the construct 101, of FIG. 1, can be utilized in a variety of configurations and/or embodiments. For example, certain embodiments of the one or more originally unsecured bridging element 122 can bridge one originally unsecured attachment element 120 to another. Certain embodiments of the one or more originally unsecured attachment element 120 and/or one or more bridging originally unsecured bridging element 122 can bridge two bony elements. Within this disclosure, the term “bony element” can include depending on context, but is not limited to, two separate bones, two portions of a broken bone, one or more vertebra, etc. Certain embodiments of the one or more originally unsecured attachment element 120 and/or the one or more originally unsecured bridging element 122 can attach one or more bone or one or more bony element to a traction or other element. For example, in one embodiment, the one or more originally unsecured bridging element 122 can include, but is not limited to, a rod that can extend between one or more originally unsecured attachment element 120. In another embodiment, such as anterior cervical spine surgery, the one or more originally unsecured bridging element 122 can include, but is not limited to, a plate that is secured by numerous originally unsecured attachment elements 120. It is envisioned that the particular surgeries as described in this disclosure are intended to be illustrative in nature, but not limiting in scope.

Within this disclosure, the position augmenting mechanism 100 and/or the construct 101 can be configured to provide and/or limit a variety of types of “context deformation” of those portions to be attached, e.g., the bony elements, etc. The two types of context deformation as described in this disclosure can include, but are not limited to discrete event context and dynamic context. Certain embodiments of the discrete event context as described with respect to FIGS. 2, 3a, and/or 3b, and 5a, 5b to 10 can entail, depending upon context, joining one or more originally unsecured bridging elements 122 in a relatively rigid or immovable manner (e.g., along three orthogonal axes), and thereupon making the connection to the originally unsecured bridging element 122 fixed and/or rigid.

Certain embodiments of the dynamic context, as described in this disclosure with respect to FIG. 4 can entail, depending upon context, situations where the construct can be allowed to change (e.g., continually or intermittently) or deform along one or more orthogonal axis. An example of where the dynamic context may be desirable is, e.g., certain cervical spine surgery, certain long bone fracture repairs. Certain embodiments of the one or more originally unsecured bridging element 122 that can provide the dynamic context 950 which can include, but is not limited to, a dynamic plate as described with respect to FIG. 4. Certain embodiments of the dynamic context can be provided, for example, at least partially between of, or alternatively outside of, the connection between the one or more originally unsecured bridging element 122 and/or the at least one of the originally unsecured context-based attachment member 120. As such, certain embodiments of the one or more originally unsecured bridging element 122 and/or the at least one of the originally unsecured context-based attachment member 120 can be configured to allow relative motion there between along one, two, or three orthogonal axes. Certain embodiments of the dynamic plate, for example, can be formed with a deformable or elastic material positioned between the one or more originally unsecured bridging element 122 and/or the at least one of the originally unsecured context-based attachment member 120, such as to allow motion between the elements 120 and 122 along at least one orthogonal axis. Such dynamic context can be provided, for example, along one, two, or more orthogonal axes. Certain embodiments of the dynamic context can be allowed or provided within the range of a fraction of a millimeter, which can be suited to stimulate a bone growth signal. Such bone growth signals may be generated, for example, when some typically sub-millimeter motion is provided between bone fragments or boney portions, while motion in other orthogonal directions can be substantially limited. For example, as described with respect to FIG. 4, the dynamic context (e.g., sub-millimeter motion) may be expected to be provided along the axial direction of the bone.

Certain embodiments of the dynamic context 950 can be provided, e.g., by allowing a designed motion between the originally unsecured bridging element 122 and the originally unsecured context-based attachment member 120, such as with some “looseness” in one or more axis, insertion of a spring or elastomeric, rubber, or other deformable member therein, etc. Certain embodiments of the dynamic plate can also be configured to achieve a bony fusion between two bony elements, an example of a construct such as described with respect to FIG. 2 can be semi-rigid in the manner that it constrains the movement of the two bony elements such as two fused vertebrae. Consider that if certain embodiments of the immobilizing construct 101 is completely rigid, it will shield the site of fusion (e.g., spinal vertebrae) from forces and/or stresses such as may be desired for certain repair or recovery such as spinal fusion.

Certain embodiments of the construct may be configured, as described with respect to FIG. 4, to provide relative motion along one or more substantially orthogonal axis (e.g., the length of the originally unsecured context-based attachment member 120) between the originally unsecured attachment element 120 relative to one or more of the originally unsecured bridging element 122. Such relative motion along one or more substantially orthogonal axes between the originally unsecured attachment element 120 relative to the one or more originally unsecured bridging element 122 can effect relative motion between bone ends, segments, bony elements, etc. It is understood by those skilled in spinal and orthopedic surgery (e.g., non-invasive, open, etc.) and recovery areas that such relative motion of bone ends can, and typically does, generate “bone growth signals” within the body to thereupon promote bone growth.

If certain embodiments of the construct 101 can be configured to be too flexible, by comparison, bone growth will be limited since the relative motion between the bone fragments or boney elements will effectively reduce or limit bone growth. For example, the site of two (e.g., broken) bone ends may be too mobile to allow for the stabile deposition of bone, since the bony elements have too much relative motion to allow for the generation of the signals to allow the bone growth. Once the bone ends are suitably maintained with some relative motion allowed there between, such as by certain embodiments of the dynamic context 950, a physiological signal (e.g., which can be generated by less than a millimeter relative motion of the bone ends) can be created within the human (or animal) with suitable motion between bone ends to create the physiological signals to grow new bone. As such, certain embodiments of the position augmenting mechanism 100 can be configured to provide a dynamic context that can allow suitable relative motion of the bone ends to provide an example of the position augmenting mechanism 100 and/or the construct 101 which can be context sensitive, such as to allow motion in one direction (e.g., axial relative to the bone, to promote signal for bone growth), but not another (e.g., orthogonal to the bone such as to limit lateral relative motion of the bone ends).

Certain embodiments of the position augmenting mechanism 100 can thereby be considered, where in its assembled state, to be the construct 101. Certain embodiments of the originally unsecured attachment element 120 should be able to be assembled with respect to the originally unsecured bridging element 122 to form the construct; and/or de-assembled relative to the one or more originally unsecured bridging element 122 as to de-assemble the construct.

FIG. 10 shows one embodiment of the position augmenting mechanism 100, as described elsewhere in this disclosure, that can utilize a position augmenting controller 97 as to allow at least partial control of certain embodiments of the position augmenting mechanism 100. Certain embodiments of the position augmenting controller can provide for at least partial sensing of positioning, and/or at least partial augmenting of positioning, of relative members such as the originally unsecured attachment element 120 and the originally unsecured bridging element 122, such as may allow for assembly or de-assembly of certain embodiments of the position augmenting mechanism 100.

Certain embodiments of the position augmenting mechanism 100 can also include certain embodiments of an adjustable originally unsecured bridging element 120, which may be adjusted, expanded, retracted, and/or otherwise adjusted remotely of the individual. Consider that certain embodiments of the originally unsecured bridging element 120 can include adjustment elements 1530 as described with respect to FIG. 11. Certain embodiments of the adjustment elements 1530 can utilize, for example, one or more of a shape memory (e.g., nitinol) based, mote-based, processor-based, nanostructural-based, or other suitable adjustment mechanism, such as generally understood by those skilled in the respective technologies, to provide adjustment of, for example, the originally unsecured bridging element 122. Within this disclosure, the term “shape memory” or “shape memory alloy” can mean depending on context, but is not limited to, being biased into a secondary position, which when actuated such as by application of an electric current, can return to its original position. One example of the shape memory alloy thereby can include, for example, nitinol.

There are a variety of mechanisms and/or techniques that can be utilized to permit adjustment of the dimensions of certain elements within certain embodiments of the position augmenting mechanism 100. Such adjustment of certain embodiments of the position augmenting mechanism 100 can occur prior to, during, or following surgery (e.g., non-invasive, open, etc.); and in certain instances such adjustment can reduce the necessity of further surgery and/or increase the effectiveness of the surgery. Certain embodiments of the adjustment elements 1530 can utilize, for example, a wireless or wired-based communication and/or processor mechanism by which it can be determined which adjustment elements 1530 should be adjusted, and by how much. Such “adjustment” of the adjustment elements 1530 can have the effect of adjusting dimensions of certain embodiments of the position augmenting mechanism 100 prior to, during, or following the surgery (e.g., non-invasive, open, etc.) in a manner that does not necessitate re-opening an incision as associated with the surgery. Such adjustment of the adjustment elements 1530 during surgery can allow for precise or other determinations of effective spans or lengths of certain embodiments of the elements of the position augmenting mechanism 100. With certain embodiments of the position augmenting mechanism 100, it may be difficult or time consuming for a surgeon or other user of the position augmenting mechanism 100 to precisely determine a suitable span or dimension upon installation. As such, a surgeon or other operating room technician, for example, can install certain embodiments of the position augmenting mechanism 100, and later during surgery or after surgery adjust the adjustment elements 1530 to the desired or suitable lengths.

Such adjustment of the adjustment elements 1530 can be even performed following surgery using, for example, a wireless communication mechanism. Additionally, following surgery and/or normal use, it may become suitable for certain embodiments of the adjustment elements 1530 to become adjusted to compensate for deformation, movement, or adjustment of the bones, vertebrae, etc., within the individual. There can be a variety of causes of such deformation, movement, or adjustment of the bones, vertebrae, etc., within the individual, many of which can be compensated for by certain embodiments of the position augmenting mechanism 100.

Consider, for example, that with certain embodiments of surgery, such as spine surgery, fractured bone surgery, and the like, it may be quite challenging to precisely space or position vertebrae, bone fragments, bony elements, etc. Additionally, it may be desired to vary the spacing between spaced or positioned vertebrae, bone fragments, bony elements, etc. following surgery based on subsequent movement of the vertebrae, bone fragments, bony elements, etc. As such it may be desirable to provide adjustment of certain components of the position augmenting mechanism 100 (and/or the associated construct).

Such adjustable embodiments of the position augmenting mechanism 100, as described with respect to FIG. 11, may thereby be considered to act as “surgical braces”, which may adjust, maintain, position, heal, or otherwise surgically handle bones, vertebrae, bone elements, bone fragments, etc. in a similar manner that dental (orthodontial) braces can position teeth within the individual's mouth. As such, the relative positioning of bones can be adjusted by actuating certain extending/retracting inserts as described with respect to FIG. 11, such as with actuation by certain embodiments of the position augmenting controller 97.

Certain embodiments of the position augmenting mechanism 100, as described with respect to FIGS. 1, 2, 3a, 3b, 4, 5a, 5b, 6a, 6b, 7a, 7b, 8a, 8b, 9a, 9b, and/or 10, for example, can provide a mechanism to enhance healing and/or treatment for an individual having, for example, spinal problems or injury; fractured, injured, or repaired bones or bony elements, etc.

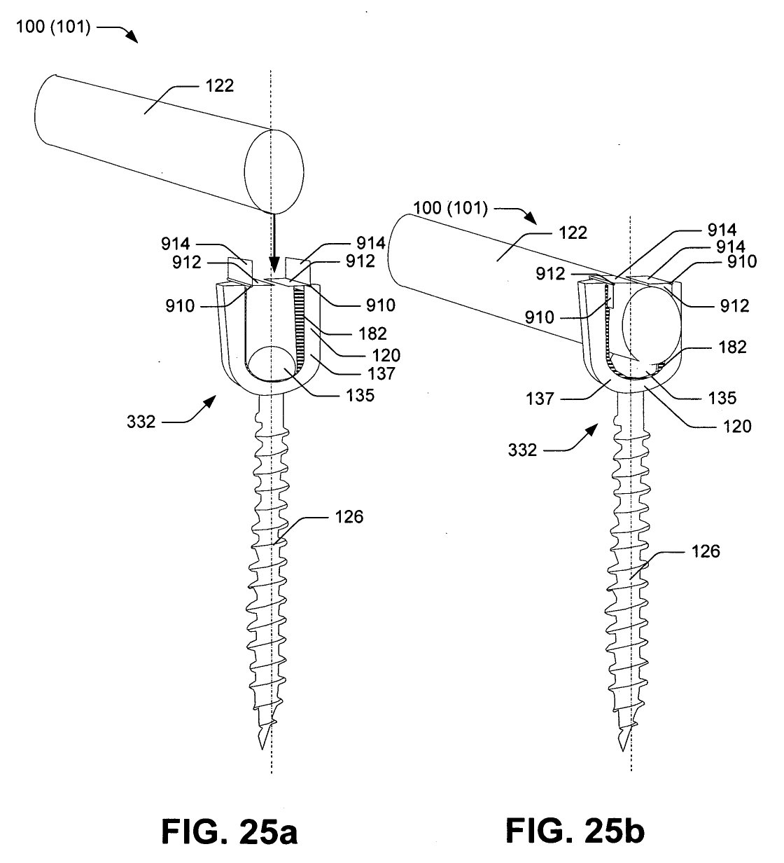

FIG. 25, which includes FIGS. 25a and 25b shows another embodiment of the position augmenting mechanism 100 that can be configured to maintain the originally unsecured attachment element 120 in a desired position with respect to the originally unsecured bridging element 122. For example, the position augmenting mechanism 100 can include a maintaining mechanism 908 including an at least one pivot 910 (two illustrated), an at least one contacting member 912 (two illustrated), and an at least one maintaining member 914 (two illustrated).

As illustrated with respect to FIGS. 25a and 25b, the at least one contacting member 912 and the at least one maintaining member 914 can each pivot some angle (e.g., approximately ninety degrees) about the at least one pivot 910 upon an occurrence of a context, such as the originally unsecured bridging element 122 being applied to the originally unsecured attachment element 120. The rotation of at least one contacting member 912 and the at least one maintaining member 914 of the of the position augmenting mechanism 100 can be between a positioning state and a maintaining state as described in this disclosure such as during assembly, or vice versa during disassembly. The originally unsecured bridging element 122 being in its positioning state can ease insertion of the originally unsecured bridging element 122 such as the rod relative to the originally unsecured bridging attachment 120, or vice versa. The originally unsecured bridging element 122 being in its maintaining state can maintain the originally unsecured bridging element 122 such as the rod relative to a desired position (such as inserted) with respect to the originally unsecured bridging attachment 120, or vice versa.

FIGS. 25a and 25b show an embodiment of the position augmenting mechanism 100 in which the at least one contacting member 912 and the at least one maintaining member 914 are formed as members attached at approximately 90 degree angles. It should be noted that certain embodiments of the at least one contacting member 912 and/or the at least one maintaining member 914 can be formed as at least one curved quarter-circle cross-section members or other suitable configuration. Certain embodiments of the maintaining member 914 can include, for example, a substantially conformable or flexible member, etc., such as to contact the originally unsecured bridging element 122 over an increased surface area, and thereby more effectively maintain the position augmenting mechanism 100 in its maintained state.

In addition, certain embodiments of the position augmenting mechanism 100 can be spring biased, such that when in its assembled state, certain embodiments of the at least one maintaining member 914 can tend to bias the originally unsecured bridging element 122 such as the rod relative into a biased engagement with the originally unsecured bridging attachment 120, or vice versa. Such biasing can be applied at a variety of locations on the at least one maintaining member 914, the at least one contacting member 912, and/or the at least one pivot 910.

The positioning state and the maintaining state between the originally unsecured bridging element 122 and the originally unsecured attachment element 120 can be as evanescent or permanent as desired or designed. For example, the at least one maintaining member 914 can include a lock member to effectively lock the suitable elements of the position augmenting mechanism 100 in an assembled or other position. Such locking of the position augmenting mechanism 100 in its maintained position can be context dependent, such as a spring-biased assembly locking the at least one maintaining member 914 in its maintained state such as described with respect to FIG. 25b. While a spring mechanism is described as one embodiment of a lock mechanism that can maintain certain embodiments of the position augmenting mechanism 100 in its maintained state, it is also envisioned that other mechanisms such as pneumatics, hydraulics, fluid mechanisms, cam mechanisms, gearing, computerized or control devices, etc. can be applied to maintain certain embodiments of the position augmenting mechanism 100 in its respective maintained state.

Certain embodiments of the at least one contacting member 912 and/or the at least one maintaining member 914 can be configured as solid plate members having a rectangular or other suitable shape. By comparison, certain embodiments of the at least one contacting member 912 and/or the at least one maintaining member 914 can be configured as interleaf fingers or the like, which may be configured to interleaf with themselves or other members. For example, certain embodiments of the at least one contacting member 912 can be configured with interleafs to interleaf with each other when in the positioning state as described with respect to FIG. 25a; and when the at least one contacting member 912 is pivoted into the maintaining state as described with respect to FIG. 25b, the individual fingers can interfit within mating recessed grooves (not shown) formed in certain embodiments of the positioning portion 137. Such mating recessed grooves can protect the at least one contacting member 912 from excessive contacts or abrasion with certain embodiments of the positioning portion 137.

Fabrication, assembly, or maintaining of certain embodiments of the position augmenting mechanism 100 can be a performed with a single person. For example, one person can hold certain embodiments of the originally unsecured bridging element 122 such as the rod in position using, e.g., a rod-holder, and then the same (or other) person can bias or displace the originally unsecured attachment element 120 into a maintaining state with respect to the originally unsecured bridging element 122, as described with respect to FIG. 25b. Certain embodiments of the position augmenting mechanism 100, as described in this disclosure, can thereby also provide a mechanism by which the position augmenting mechanism 100 can maintain and/or lock the originally unsecured attachment element 120 relative to the originally unsecured bridging element 122 such as the rod or plate.

Another embodiment of the position augmenting mechanism 100 is described with respect to FIG. 26, which includes FIGS. 26a and 26b, which includes maintaining biased detents 915. Certain embodiments of the maintaining biased detents 915 can be configured with spring or other biasing such that retraction or expansion can be permitted such as to allow the originally unsecured bridging element 122 to travel from the positioning state to the maintaining state with respect to the originally unsecured attachment element 120. When the maintaining biased detent(s) 915 is positioned or displaced into its expanded state, certain embodiments of the originally unsecured bridging element 122 can travel with respect to the originally unsecured attachment element 120 such as between the positioning state and the maintaining state. By comparison, when the maintaining biased detent(s) 915 is in its extended position, certain embodiments of the originally unsecured bridging element 122 can be limited from travel with respect to the originally unsecured attachment element 120 (such as to be maintained in the maintaining state). The biasing effect of certain embodiments of the maintaining biased detents 915 can be selected such as to be easily overcome such as by a person applying a suitable overcoming force, but may also be sufficient to maintain the position augmenting mechanism 100 in its maintained state as described in this disclosure.

FIG. 27, including FIGS. 27a and 27b, show another embodiment of the position augmenting mechanism 100 that can include another embodiment of the maintaining biased detents 942, which are configured as plates that in general are pivoted and biased into some generally downward angled configuration. Certain embodiments of the maintaining biased detents 942 can be configured with spring or other biasing such that retraction or expansion of the plate can be permitted such as to allow the originally unsecured bridging element 122, such as the rod, to travel from the positioning state to the maintaining state with respect to the originally unsecured attachment element 120. When the maintaining biased detent(s) 942 is positioned or displaced into its expanded state, certain embodiments of the originally unsecured bridging element 122 can travel with respect to the originally unsecured attachment element 120 such as between the positioning state and the maintaining state. By comparison, when the maintaining biased detent(s) 942 is in its extended position, certain embodiments of the originally unsecured bridging element 122 can be biased against travel with respect to the originally unsecured attachment element 120 (such as to be maintained in the maintaining state). The biasing effect of certain embodiments of the maintaining biased detents 942 can be selected such as to be overcome such as by a person applying a suitable overcoming force, but may also be sufficient to maintain the position augmenting mechanism 100 in its maintained state as described in this disclosure.

As such, the embodiment of the position augmenting mechanism 100, as described with respect to FIGS. 27a and 27b, for example, can secure, maintain, and/or lock the originally unsecured bridging element 122 secured with respect to the originally unsecured attachment element 120 using a variety of mechanisms that can be normally utilized by a single physician, or other medical personnel.

Certain embodiments of the position augmenting mechanism 100 can also include the securing or locking mechanism 960, which can be configured as a screw cap, which can be matingly threaded with portions of the originally unsecured attachment element 120 to maintain or lock the originally unsecured bridging element 122 secured with respect to the originally unsecured attachment element 120. Other embodiments of the position augmenting mechanism 100 can include a threaded, glued, fastened, or other mechanism that can be configured to act as alternative embodiments of the securing or locking mechanism 960 to lock or secure the originally unsecured bridging element 122 in a desired position with respect to the originally unsecured attachment element 120. Certain embodiments of the attachment-enhancing member 182, as illustrated in FIG. 27a and described elsewhere in this disclosure, can be threaded or otherwise configured to allow a mating or secure engagement with certain embodiments of the securing or locking mechanism 960.

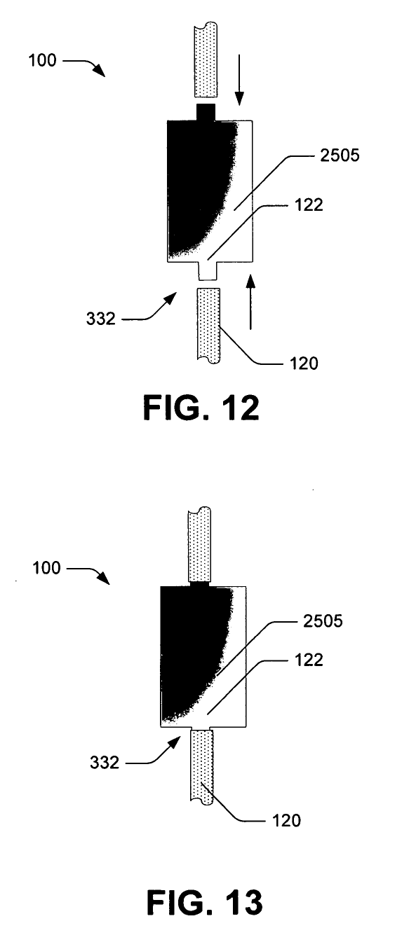

Certain embodiments of the position augmenting mechanism 100 can provide for attachment of a fluid handling mechanism, such as a shunt 2505 as described respectively with respect to FIGS. 12 and 13 in the respective unassembled and assembled states. Certain embodiments of the position augmenting mechanism 100 can thereby be utilized to include a shunt valve, such as for hydrocephalus. Certain embodiments of the position augmenting mechanism can also be configured, for example, for heart valves, stent (tubing or valve, with low pressure, valve closes, with high pressure, valve opens), etc. (not illustrated). The mechanism of attaching or assembling the embodiments of the position augmenting mechanism 100 as described with respect to FIGS. 12 and 13, for example, can be similar to that as described in this disclosure with respect to FIGS. 1, 2, 3a, 3b, 4, 5a, 5b, 6a, 6b, 7a, 7b, 8a, 8b, 9a, 9b, 10, and/or 11. Certain embodiments of the position augmenting mechanism 100 can, depending on context, thereby involve the at least one originally unsecured attachment element 120 being configurable to improve a relative positioning with respect to an at least one originally unsecured bridging element 122 when the former is not in the correct range of desired position(s); and the at least one originally unsecured attachment element being configurable to maintain a relative positioning with respect to the at least one originally unsecured bridging element when the former is in the correct range of desired position(s).

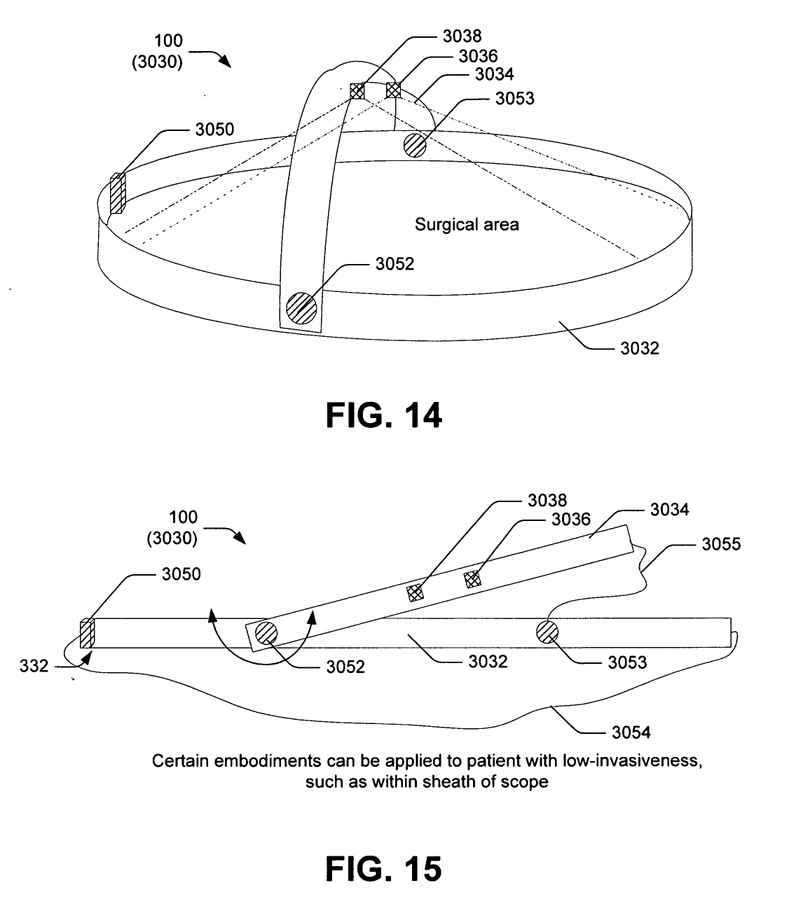

Certain embodiments of the position augmenting mechanism 100, as described with respect to respective FIGS. 14 and 15 in its respective assembled state and un-assembled state, can be configured to provide for a surgical theater 3030. Certain embodiments of the position augmenting mechanism 100 can thereby be configured as a surgical theater that when assembled, can assume a dome or similar configuration which can deflect, support, and/or maintain the visceral wall of the individual over the organs at least partially forming such cavities as the abdomen cavity and/or the thoracic cavity (chest). As such, certain embodiments of the context of the position augmenting mechanism 100 can be configured, when assembled, to support such walls as the abdominal wall and/or the thoracic wall away from the corresponding organs, thereby providing the surgeon or other medical individual considerable access to the organs within those cavities.

One embodiment of the position augmenting mechanism 100, which can be configured to provide a surgical theater 3030 as described with respect to FIG. 14, can include but is not limited to, a stabilizing member 3032, a maintaining member 3034, a light provider 3036, and an image capturer 3038. In certain embodiments, the stabilizing member 3032 can maintain the surgical theater 3030 in a stable position within the individual, such as the patient, during a surgery or an operation. In certain embodiments, the maintaining member 3034 can be configured to support such walls as the abdominal wall and/or the thoracic wall away from the corresponding organs, which may thereby provide the surgeon or other medical individual considerable accessed to the organs within those cavities.

Certain embodiments a light provider 3036 can be configured to provide light within the abdominal cavity and/or thoracic cavity, such that the surgeon or other operating room attendant can view within the abdominal cavity and/or thoracic cavity utilizing, e.g., scopes, cameras, and/or the image capturer 3038, etc. Certain embodiments of the image capture 3038 can be configured as the scopes, cameras, and/or imager such that the surgeon or operating room attendant can view within the abdominal cavity and/or thoracic cavity, in certain embodiments utilizing wireless technology such as can be displayed outside of the individual. While the light provider 3036 and the image capturer 3038 are illustrated, with respect to FIGS. 14 and 15, as being secured to the maintaining member 3034, it is envisioned that one or more of the members 3036 and/or 3038 can be situated at other functional locations of the surgical theater.

The configuration of certain embodiments of the surgical theater 3030 can vary depending upon usage, design, desired duration, repetition of surgery, etc. For example, it may be envisioned that the surgical theater may be used on a one-time basis, such as during normal operations. By comparison, certain patients within intensive care units, for example, may undergo repeated surgeries in which case may be desired to keep the surgical theater 3030 within the patient for the duration that the patient is within intensive care, or some other duration. Certain embodiments of the surgical theater 3030 may even be configured as substantially permanent within the individual such as with chronically ill individuals.

Certain embodiments of the position augmenting mechanism 100, such as that described with respect to the surgical theater 3030 of FIG. 14, can be applied in its unexpanded form via a sheath in a scope device such as an endoscope as described with respect to FIG. 15. For instance, certain embodiments of the surgical theater can be inserted in its un-assembled state into the sheath of the scope, and the un-assembled surgical theater can thereupon be applied to within the abdominal cavity and/or thoracic cavity, and at which time it may be expelled from the sheath. In certain embodiments, the surgical theater 3030 can thereupon be expanded, into a state similar to as described with respect to FIG. 14. Certain embodiments of the surgical theater 3030 can be assembled, for example, by assembling the stabilizing member 3032 as well as assembling the maintaining member 3034. For example, certain embodiments of the stabilizing member 3032 can be assembled by allowing a retraction member 3050 to retract a retracting member 3054 (e.g., a string, rubber or plastic grooved or toothed member, etc.). By the retraction member 3050 retracting the retracting member 3054, the stabilizing member changes it configuration from that shown in FIG. 15 to that shown in FIG. 14, such that the stabilizing member can be a self-supporting and stable member. By the retraction member 3050 extending the retracting member 3054, the stabilizing member can change its configuration from that shown in FIG. 14 to that shown in FIG. 15, such that the stabilizing member can be applied to an individual such as via a sheath of a scope.

Certain embodiments of the maintaining member 3034 can be assembled into its operational state (e.g., when the stabilizing member 3032 is in its assembled position) by rotating a retracting member 3054 that rotatably connects the maintaining member 3034 and the stabilizing member 3032 until the maintaining member 3034 at its junction with the stabilizing member 3032 is substantially perpendicular to the stabilizing member 3032. In this position, when inside of the individual, certain embodiments of the surgical theater can support the thoracic wall or abdominal wall. In addition, certain embodiments of a retraction member 3050 can retract a retracting member 3054 such that the maintaining member 3034 can form into a supportive shape similar to as described with respect to FIG. 14. Certain embodiments of the retracting member 3054 can include, but is not limited to, a string, rubber or plastic grooved or toothed member, etc. If the retraction member 3050 allows the retracting member 3054 to extend, then the surgical theater can form into the configuration such as described with respect to FIG. 15, in which the surgical theater can be applied to within the individual utilizing a scope, etc.

By allowing certain embodiments of the position augmenting mechanism 100 to be applied within a sheath of an endoscope, the invasiveness of the surgical techniques utilizing certain embodiments of the position augmenting mechanism 100 can thereby be reduced as compared with creating larger incisions, etc. As such, certain embodiments of position augmenting mechanism 100 can thereby be utilized to realize decreased invasiveness surgical theaters built within the cavity of a body.

Certain embodiments of the surgical theater 3030 can thereupon be expanded, such as within the abdominal cavity and/or thoracic cavity of the individual. Assembly can be provided using a variety of mechanisms. For example, as illustrated with respect to FIG. 14, the stabilizing member 3032 can be configured to form a stable platform, such by bringing the ends of the stabilizing member can be closed together to form, e.g., a circle, oval, or some other stable configuration against turning-over, etc. In certain embodiments, the maintaining member 3034 can thereupon be extended such as to extend the abdominal wall and/or the thoracic wall away from the respective organs, etc., such as to increase the dimensions of the respective abdominal cavity and/or thoracic cavity. By extending the abdominal wall and/or thoracic wall away from the organs of the patient, etc., the organs contained within the respective cavity can be more clearly visible.

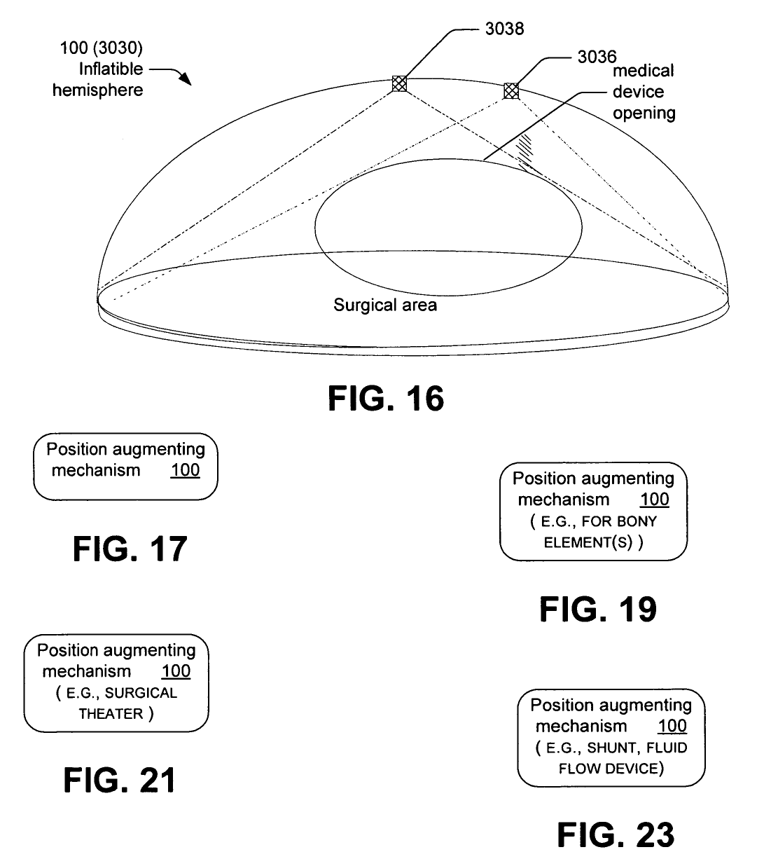

Certain embodiments of the surgical theater 3030 can thereby be temporary, such as may be utilized during endoscopic surgery. Consider that, for example, with certain embodiments of abdominal or thoracic surgery, lighting can be a problem. As such, certain embodiments of the surgical theater 3030 can provide a lighting frame such as to allow the surgeon, and other operating room attendants, to view the surgery from a distance. While this disclosure describes a number of illustrative mechanisms (which are not intended to be limiting scope) to create a structural support frame (and optional light source/camera) within the abdominal cavity and/or the thoracic cavity that may act as the surgical theater 3030, it may be envisioned that other mechanisms can be utilized to provide a surgical theater. For example, certain embodiments of the position augmenting mechanism 100 as described with respect to FIG. 16 can be inflatable (e.g., such as to form a hemidome when inflated), which can also be inserted in the sheath of the scope such as to also provide a low-invasive surgical theater. Certain embodiments of the hemidome, for example, can be configured with recesses, apertures, etc., one of which is illustrated in FIG. 16, such as to allow physicians or other medical individuals to apply surgical tools, medical devices, scopes, etc. there through to a surgical area in a manner such as would be expected during surgery, etc. Such position augmenting mechanisms 100 configured as a variety of surgical theaters can be produced using a variety of technologies. The embodiments of the surgical theaters as described with respect to FIGS. 14-16 may be intended to be illustrative in nature but not limiting in scope.

### 2. Certain Embodiments of the Position Augmenting Controller

This disclosure describes a number of embodiments of the position augmenting controller 97 as described with respect to FIG. 10, which are intended to control operations of the position augmenting mechanism 100 to effectively assemble and/or de-assemble the construct. As such, certain embodiments of the position augmenting mechanism 100 can operate without, and/or with little interaction from, the position augmenting controller 97. By comparison, certain embodiments of the position augmenting mechanism 100 can operate with considerable input from, and/or entirely utilizing input from, the position augmenting controller 97.

While certain embodiments of the context-based assembly portion 332 of the originally unsecured attachment element 120 may not utilize processors either: a) to sense the relative position of the originally unsecured bridging element 122 and/or b) to attach to or de-attach from the originally unsecured bridging element 122. As such, this portion of the disclosure describes certain embodiments of the position augmenting controller 97 for those embodiments of the position augmenting mechanism 100 that are configured to utilize the position augmenting controller.

Certain embodiments of the position augmenting mechanism 100 can thereby include the position augmenting controller 97; while other embodiments of the position augmenting mechanism may not include utilizing certain embodiments of the position augmenting controller. For instance, certain embodiments of the position augmenting mechanism 100 including the position augmenting controller 97, which are largely microprocessor-based, can provide for largely automated attachment or assembly of the position augmenting mechanism 100. For instance, certain embodiments of the position augmenting controller 97 can be configured to upon actuation and/or deactuation, provide for attachment of the originally unsecured attachment element 120 with respect to the originally unsecured bridging element 122. By comparison, certain embodiments of the position augmenting mechanism 100 which can be attached or assembled utilizing largely manual techniques may not utilize the position augmenting mechanism 100 as described in this disclosure of with respect to FIGS. 1-9. FIG. 10 thereby can show a block diagram of certain respective embodiments of the position augmenting mechanism 100 that can include the position augmenting controller 97 to either control the securing of the elements within the position augmenting mechanism, or some other related operation such as sensing relative positioning of the originally unsecured attachment element 120 with respect to the originally unsecured bridging element 122.

Certain embodiments of the position augmenting mechanism 100 thereby can include, but are not limited to, any particular configuration of the position augmenting controller 97. Certain embodiments of the position augmenting controller 97 can be computer based, controller based, mote based, cellular telephone-based, and/or electronics based. Certain embodiments of the position augmenting controller can be segmented into modules, and can utilize a variety of wireless communications and/or networking technologies to allow information, data, etc. to be transferred to the various distinct portions or embodiments of the position augmenting mechanism 100. Certain embodiments of the position augmenting controller 97 can be configured as a unitary or stand alone device.

Certain embodiments of the position augmenting controller 97 can vary as to their automation, complexity, and/or sophistication; and can be utilized to control, setup, establish, and/or maintain communications between a number of communicating devices. As described within this disclosure, multiple ones of the different embodiments of the position augmenting mechanism 100 can transfer information or data relating to the communication link to or from a remote location and/or some intermediate device as might be associated with communication, monitoring and/or other activities.

Certain embodiments of the position augmenting controller 97, as well as certain embodiments of the position augmenting mechanism 100 (in general), can utilize distinct firmware, hardware, and/or software technology. For example, mote-based technology, microprocessor-based technology, microcomputer-based technology, general-purpose computer technology, specific-purpose computer technology, Application-Specific Integrated Circuits, and/or a variety of other computer technologies can be utilized for certain embodiments of the position augmenting controller 97, as well as certain embodiments of the position augmenting mechanism 100.

Certain embodiments of the position augmenting controller 97 can as described with respect to FIG. 10 can include depending on context a processor 803 such as a central processing unit (CPU), a memory 807, a circuit or circuit portion 809, and an input output interface (I/O) 811 that may include a bus (not shown). Certain embodiments of the position augmenting controller 97 of the position augmenting mechanism 100 can include and/or be a portion of a general-purpose computer, a specific-purpose computer, a microprocessor, a microcontroller, a personal display assistant (PDA), a cellular phone, a wireless communicating device, a hard-wired phone, and/or any other known suitable type of communications device, computer, and/or controller that can be implemented in hardware, software, electromechanical devices, and/or firmware. Certain embodiments of the processor 803, as described with respect to FIG. 10, can perform the processing and arithmetic operations for certain embodiments of the position augmenting controller 97 of the position augmenting mechanism 100. Certain embodiments of the position augmenting controller 97 of the position augmenting mechanism 100 can control the signal processing, database querying and response, computational, timing, data transfer, and other processes associated with certain embodiments of the position augmenting controller 97 of the position augmenting mechanism 100.

Certain embodiments of the memory 807 of the position augmenting controller 97 can include a random access memory (RAM) and/or read only memory (ROM) that together can store the computer programs, operands, and other parameters that control the operation of certain embodiments of the position augmenting controller 97 of the position augmenting mechanism 100. The memory 807 can be configurable to contain information obtained, retained, or captured by that particular position augmenting controller 97 of the position augmenting mechanism 100.

Certain embodiments of the bus can be configurable to provide for digital information transmissions between the processor 803, circuits 809, memory 807, I/O 811, and/or the image memory or storage device (which may be integrated or removable). In this disclosure, the memory 807 can be configurable as RAM, flash memory, semiconductor-based memory, of any other type of memory that can be configurable to store data pertaining to images. The bus also connects I/O 811 to the portions of certain embodiments of the position augmenting controller 97 of either the position augmenting mechanism 100 that can either receive digital information from, or transmit digital information to other portions of the position augmenting mechanism 100, or other systems and/or networking components associated with.

Certain embodiments of the position augmenting controller 97 of the position augmenting mechanism 100, as described with respect to FIG. 10, can include a transmitter portion (not shown) that can be either included as a portion of certain embodiments of the position augmenting controller 97 of the position augmenting mechanism 100. Certain embodiments of the position augmenting controller 97 can alternately be provided as a separate unit (e.g., microprocessor-based). In certain embodiments, the transmitter portion can transmit image information between certain embodiments of the position augmenting controller 97 of the position augmenting mechanism 100.

Certain embodiments of the position augmenting controller 97 of the position augmenting mechanism 100 as described with respect to FIG. 10 can include an operation altering portion (not shown) that can be either included as a portion of certain embodiments of the position augmenting controller 97 of the position augmenting mechanism 100, or alternately can be provided as a separate unit (e.g., microprocessor-based).

Certain embodiments of the memory 807 can provide one example of a memory storage portion. In certain embodiments, the monitored value includes but is not limited to: a percentage of the memory 807, an indication of data that is or can be stored in the memory 807, or for data storage or recording interval. To provide for overflow ability for the memory 807 of certain embodiments of the position augmenting controller 97 of the position augmenting mechanism 100, a secondary storage device can be operably coupled to the memory 807 to allow a controllable transmitting of memory data from certain embodiments of the position augmenting controller 97 of the position augmenting mechanism 100 when the monitored value of data or other information within the memory 807 exceeds a prescribed value. The prescribed value can include, e.g., some percentage amount or some actual amount of the value.

In certain embodiments, a secondary communication link can be established between the certain embodiments of the position augmenting controller 97 of the position augmenting mechanism 100. The secondary communication link can be structured similar to as a communication link, or alternatively can utilize network-based computer connections, Internet connections, etc. to provide information and/or data transfer between certain embodiments of the position augmenting controller 97 of the position augmenting mechanism 100.

In certain embodiments of the position augmenting controller 97 of the position augmenting mechanism 100, the particular elements of certain embodiments of the position augmenting controller 97 of the position augmenting mechanism 100 (e.g., the processor 803, the memory 807, the circuits 809, and/or the I/O 811) can provide a monitoring function to convert raw data as displayed by an indicator. A monitoring function as provided by certain embodiments of the position augmenting controller 97 of the position augmenting mechanism 100 can be compared to a prescribed limit, such as whether the number of images contained in the memory 807, the amount of data contained within the memory 807, or some other measure relating to the memory is approaching some value. The limits to the value can, in different embodiments, be controlled by the user or the manufacturer of certain embodiments of the position augmenting controller 97 of the position augmenting mechanism 100. In certain embodiments, the memory 807 can store such information as data, information, displayable information, readable text, motion images, video images, and/or audio images, etc.

In certain embodiments, the I/O 811 provides an interface to control the transmissions of digital information between each of the components in certain embodiments of the position augmenting controller 97 of the position augmenting mechanism 100. The I/O 811 also provides an interface between the components of certain embodiments of the position augmenting controller 97 of the position augmenting mechanism 100. The circuits 809 can include such other user interface devices as a display and/or a keyboard. In other embodiments, the position augmenting controller 97 of the position augmenting mechanism 100 can be constructed as a specific-purpose computer such as an application-specific integrated circuit (ASIC), a microprocessor, a microcomputer, or other similar devices.

### 3. Certain Embodiments of the Position Augmenting Mechanism with Relevant Flowcharts

Within the disclosure, flow charts of the type described in this disclosure apply to method steps as performed by a computer or controller as could be contained within certain embodiments of the position augmenting mechanism 100, as described in this disclosure. Additionally, the flow charts as described in this disclosure apply operations or procedures that can be performed entirely and/or largely utilizing mechanical devices, electromechanical devices, or the like, such as certain embodiments of the position augmenting mechanism 100 as described in this disclosure. The flow charts can also apply to apparatus devices, such as an antenna or a node associated therewith that can include, e.g., a general-purpose computer or specialized-purpose computer whose structure along with the software, firmware, electro-mechanical devices, and/or hardware, can perform the process or technique described in the flow chart.

FIG. 17 shows one embodiment of the position augmenting mechanism 100 that can act to augment an originally unsecured bridging element relative to an originally unsecured attachment element. There can be a variety of embodiments of the position augmenting mechanism 100 that can be configured to operate as, but is not limited to, the surgical rod(s) or plate(s), as described with respect to FIGS. 1, 2, 3a, 3b, 4, 5a, 5b, 6a, 6b, 7a, 7b, 8a, 8b, 9a, 9b, 10, and/or 11 that can treat, for example, bony elements that can include, but are not limited to, bones, bone fragments, vertebrae, etc. There can be variety of embodiments of the position augmenting mechanism 100 that can be configured to operate as, but is not limited to, the shunt or other fluid-flow-based device, as described with respect to FIGS. 12 and/or 13. There can be a variety of embodiments of the position augmenting mechanism 100 that can be configured as, but is not limited to, a surgical theater or other internal structure such as a heartfelt, as described with respect to FIGS. 14, 15, and/or 16.

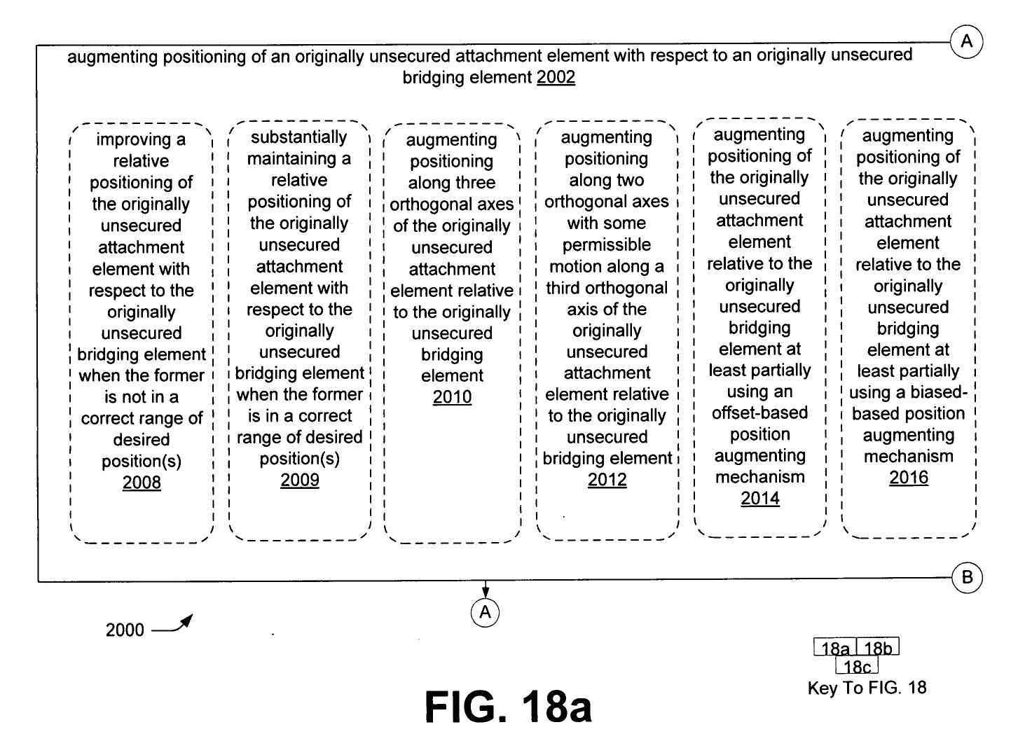

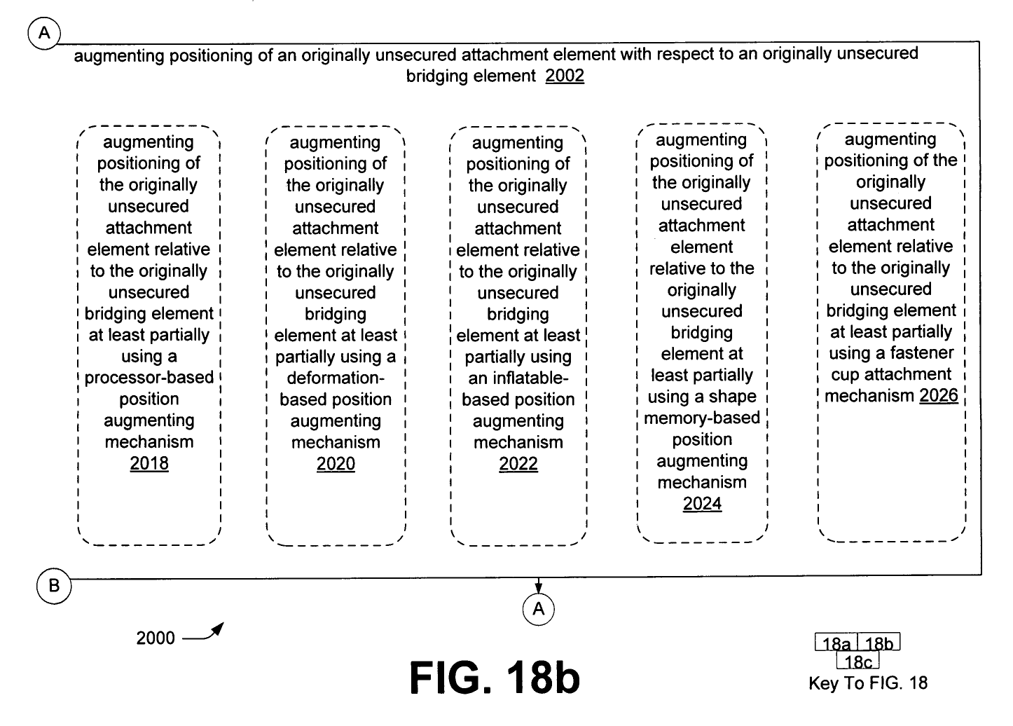

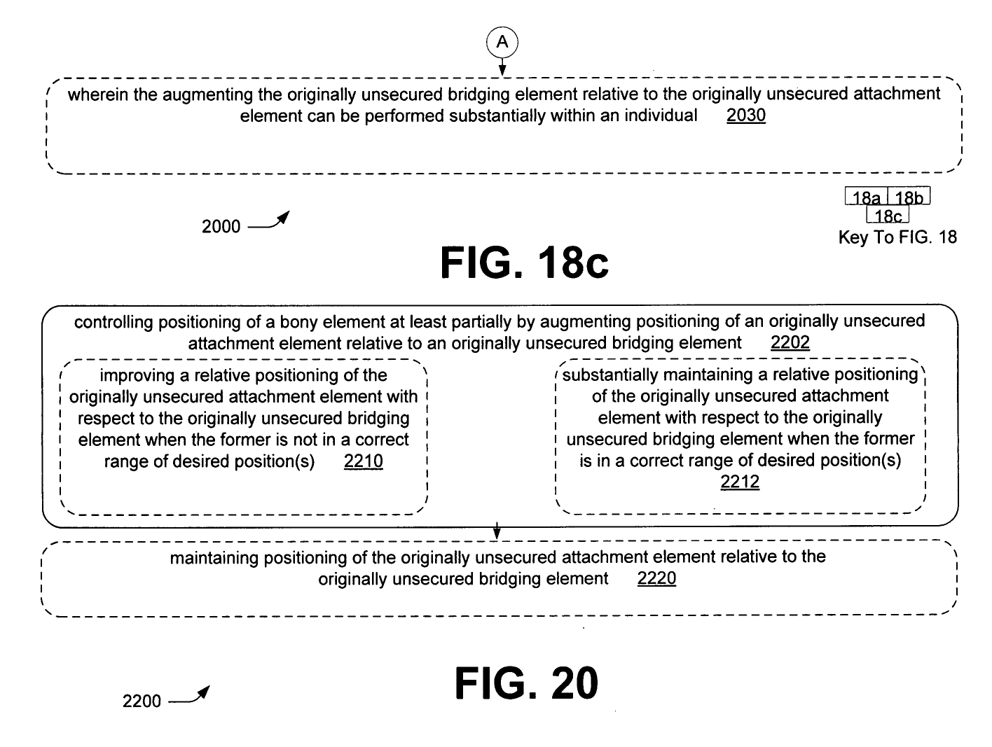

FIG. 18 shows one embodiment of the position augmenting mechanism 100 such as described with respect to, but not limited to, FIGS. 1 to 10, and elsewhere in this disclosure. One embodiment of a high-level flowchart of an attachment mechanism 2000 is described with respect to FIG. 18 and can include, but is not limited to, operation 2002 and/or optional operation 2030. One embodiment of operation 2002 can include, but is not limited to, operations 2008, 2009, 2010, 2012, 2014, 2016, 2018, 2020, 2022, 2024, and/or 2026. The high-level flowchart of FIG. 18 should be considered in combination with the embodiments of the position augmenting mechanism 100, as described with respect to FIG. 17, and elsewhere in this disclosure. One embodiment of operation 2002 can include, but is not limited to, augmenting positioning of an originally unsecured attachment element with respect to an originally unsecured bridging element. For example, there can be a variety of embodiments of the position augmenting mechanism 100 that can be configured to operate as, but is not limited to, the surgical rod(s) or plate(s), as described with respect to FIGS. 1, 2, 3a, 3b, 4, 5a, 5b, 6a, 6b, 7a, 7b, 8a, 8b, 9a, 9b, 10, and/or 11 that can treat, for example, bony elements that can include, but are not limited to, bones, bone fragments, vertebrae, etc. There can be variety of embodiments of the position augmenting mechanism 100 that can be configured to operate as, but is not limited to, the shunt or other fluid-flow-based device, as described with respect to FIGS. 12 and/or 13. There can be a variety of embodiments of the position augmenting mechanism 100 that can be configured as, but is not limited to, a surgical theater or other internal structure such as a heartfelt, as described with respect to FIGS. 14, 15, and/or 16. One embodiment of the augmenting positioning of an originally unsecured attachment element with respect to an originally unsecured bridging element of operation 2002 can include operation 2008, that can include, but is not limited to, improving a relative positioning of the originally unsecured attachment element with respect to the originally unsecured bridging element when the former is not in a correct range of desired position(s). For example, allowing the originally unsecured attachment element to be positioned or adjusted relative to the originally unsecured bridging element such as to permit assembly of certain embodiments of the position augmenting mechanism. One embodiment of the augmenting positioning of an originally unsecured attachment element with respect to an originally unsecured bridging element of operation 2002 can include operation 2009, that can include, but is not limited to, substantially maintaining a relative positioning of the originally unsecured attachment element with respect to the originally unsecured bridging element when the former is in a correct range of desired position(s). For example, allowing the originally unsecured attachment element to be maintained in position relative to the originally unsecured bridging element such as to permit assembly of certain embodiments of the position augmenting mechanism. With such maintaining, certain embodiments of the position augmenting mechanism 100 can involve applying another device, such as the securing or locking mechanism 960 as described with respect to FIGS. 27a and 27b, to secure or lock the elements 120 and 122 in position relative to each other. One embodiment of the augmenting positioning of an originally unsecured attachment element with respect to an originally unsecured bridging element of operation 2002 can include operation 2010, that can include, but is not limited to, augmenting positioning along three orthogonal axes of the originally unsecured attachment element relative to the originally unsecured bridging element. For example, the originally unsecured attachment element can be augmented position away with respect to the originally unsecured bridging element along three orthogonal axes. Consider that it might be desired to fuse vertebrae for cervical, thoracic, and lumbar spinal support. One embodiment of the augmenting positioning of an originally unsecured attachment element with respect to an originally unsecured bridging element of operation 2002 can include operation 2012, that can include, but is not limited to, augmenting positioning along two orthogonal axes with some permissible motion along a third orthogonal axis of the originally unsecured attachment element relative to the originally unsecured bridging element. For example, positioning of the originally unsecured attachment element with respect to the originally unsecured bridging element can be augmented along two orthogonal axes, and at least some motion can be allowed along the third orthogonal axis. Consider, for example, that sub-millimeter motion can be provided for bone fracture repair. One embodiment of the augmenting positioning of an originally unsecured attachment element with respect to an originally unsecured bridging element of operation 2002 can include operation 2014, that can include, but is not limited to, augmenting positioning of the originally unsecured attachment element relative to the originally unsecured bridging element at least partially using an offset-based position augmenting mechanism. For example, certain embodiments of the offset-based position augmenting mechanism can utilize, but is not limited to, one or more cam as described with respect to FIGS. 5a and 5b. One embodiment of the augmenting positioning of an originally unsecured attachment element with respect to an originally unsecured bridging element of operation 2002 can include operation 2016, that can include, but is not limited to, augmenting positioning of the originally unsecured attachment element relative to the originally unsecured bridging element at least partially using a biased-based position augmenting mechanism. For example, certain embodiments of the biased-based position augmenting mechanism can utilize, but is not limited to, one or more cam as described with respect to FIGS. 6a and 6b. One embodiment of the augmenting positioning of an originally unsecured attachment element with respect to an originally unsecured bridging element of operation 2002 can include operation 2018, that can include, but is not limited to, augmenting positioning of the originally unsecured attachment element relative to the originally unsecured bridging element at least partially using a processor-based position augmenting mechanism. For example, certain embodiments of the processor based position augmenting mechanism can utilize, but is not limited to, one or more spring or biasing mechanism as described with respect to FIG. 10. One embodiment of the augmenting positioning of an originally unsecured attachment element with respect to an originally unsecured bridging element of operation 2002 can include operation 2020, that can include, but is not limited to, augmenting positioning of the originally unsecured attachment element relative to the originally unsecured bridging element at least partially using a deformation-based position augmenting mechanism. For example, certain embodiments of the deformation-based position augmenting mechanism can utilize, but is not limited to, one or more crimps as described with respect to FIGS. 3a, 3b, 6a, 6b, 7a and 7b. One embodiment of the augmenting positioning of an originally unsecured attachment element with respect to an originally unsecured bridging element of operation 2002 can include operation 2022, that can include, but is not limited to, augmenting positioning of the originally unsecured attachment element relative to the originally unsecured bridging element at least partially using an inflatable-based position augmenting mechanism. For example, certain embodiments of the inflatable-based position augmenting mechanism can utilize, but is not limited to, one or more balloons as described with respect to FIGS. 8a and 8b. One embodiment of the augmenting positioning of an originally unsecured attachment element with respect to an originally unsecured bridging element of operation 2002 can include operation 2024, that can include, but is not limited to, augmenting positioning of the originally unsecured attachment element relative to the originally unsecured bridging element at least partially using a shape memory-based position augmenting mechanism. For example, certain embodiments of the shape memory-based position augmenting mechanism can utilize, but is not limited to, one or more shape memory-based materials such as nitinol, as described with respect to FIGS. 9a and 9b. One embodiment of the augmenting positioning of an originally unsecured attachment element with respect to an originally unsecured bridging element of operation 2002 can include operation 2026, that can include, but is not limited to, augmenting positioning of the originally unsecured attachment element relative to the originally unsecured bridging element at least partially using a fastener cup attachment mechanism. For example, certain embodiments of the position augmenting mechanism can utilize, but is not limited to, one or more fastener cup attachment mechanisms as described with respect to FIGS. 9a and 9b. One embodiment of operation 2030 can include, but is not limited to, wherein the augmenting the originally unsecured attachment element relative to the originally unsecured bridging element can be performed substantially within an individual. For example, the augmenting of certain embodiments of the position augmenting mechanism be performed within the individual, such as a human or animal. The order of the operations, methods, mechanisms, etc. as described with respect to FIG. 18 is intended to be illustrative in nature, and not limited in scope.

FIG. 19 shows one embodiment of the position augmenting mechanism 100 that can act to augment an originally unsecured bridging element relative to an originally unsecured attachment element. There can be a variety of embodiments of the position augmenting mechanism 100 that can be configured to operate as, but is not limited to, the surgical rod(s) or plate(s), as described with respect to FIGS. 1, 2, 3a, 3b, 4, 5a, 5b, 6a, 6b, 7a, 7b, 8a, 8b, 9a, 9b, 10, and/or 11 that can treat, for example, bony elements that can include, but are not limited to, bones, bone fragments, vertebrae, etc.

FIG. 20 shows one embodiment of the position augmenting mechanism 100 such as described with respect to, but not limited to, FIGS. 1 to 10, and elsewhere in this disclosure. One embodiment of a high-level flowchart of an attachment mechanism 2200 is described with respect to FIG. 20 and can include, but is not limited to, operation 2202 and optional operation 2220. Certain embodiments of the operation 2202 can include, but is not limited to, operations 2210 and 2212. The high-level flowchart of FIG. 20 should be considered in combination with the embodiments of the position augmenting mechanism 100, as described with respect to FIG. 19, and elsewhere in this disclosure. One embodiment of operation 2202 can include, but is not limited to, controlling positioning of a bony element at least partially by augmenting positioning of an originally unsecured attachment element relative to an originally unsecured bridging element. Certain embodiments of operation 2220 can include, but is not limited to, maintaining positioning of the originally unsecured attachment element relative to the originally unsecured bridging element. For instance, either element 120 or 122 can be secured with respect to the other element by temporarily or more permanently maintaining, such as tending to hold one element in position with respect to the other element. Certain embodiments of the controlling positioning of a bony element at least partially by augmenting positioning of an originally unsecured attachment element relative to an originally unsecured bridging element of operation 2202 can include operation 2210 that can include, but is not limited to, improving a relative positioning of the originally unsecured attachment element with respect to the originally unsecured bridging element when the former is not in a correct range of desired position(s). For example, repositioning and/or securing the bony element relative to the originally unsecured bridging element. Certain embodiments of the controlling positioning of a bony element at least partially by augmenting positioning of an originally unsecured attachment element relative to an originally unsecured bridging element of operation 2202 can include operation 2212 that can include, but is not limited to, substantially maintaining a relative positioning of the originally unsecured attachment element with respect to the originally unsecured bridging element when the former is in a correct range of desired position(s). For example, maintaining the relative positioning of the originally unsecured attachment element. As such, certain embodiments of the position augmenting mechanism 100 as described with respect to FIGS. 1, 2, 3a, 3b, 4, 5a, 5b, 6a, 6b, 7a, 7b, 8a, 8b, 9a, 9b, 10, and/or 11 can control positioning of a bony element at least partially by augmenting positioning of an originally unsecured attachment element relative to an originally unsecured bridging element. The order of the operations, methods, mechanisms, etc. as described with respect to FIG. 20 is intended to be illustrative in nature, and not limited in scope.

FIG. 21 shows one embodiment of the position augmenting mechanism 100 that can be configured as a surgical theater whose position can be augmented. There can be a variety of embodiments of the position augmenting mechanism 100 that can be configured as, but is not limited to, a surgical theater or other internal structure such as a heartfelt, as described with respect to FIGS. 14, 15, and/or 16.

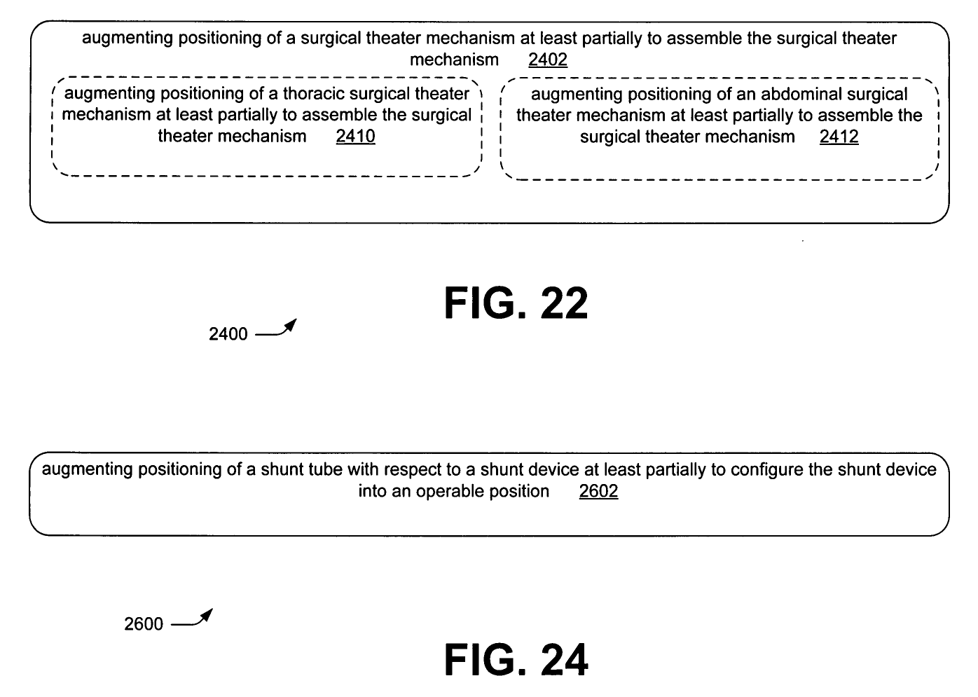

FIG. 22 shows one embodiment of the position augmenting mechanism 100 such as described with respect to, but not limited to, FIGS. 1 to 10, and elsewhere in this disclosure. One embodiment of a high-level flowchart of an attachment mechanism 2400 is described with respect to FIG. 22 and can include, but is not limited to, operation 2402. One embodiment of operation 2402 can include, but is not limited to, operations 2410 and 2412. The high-level flowchart of FIG. 22 should be considered in combination with the embodiments of the position augmenting mechanism 100, as described with respect to FIG. 21, and elsewhere in this disclosure. One embodiment of operation 2402 can include, but is not limited to, augmenting positioning of a surgical theater mechanism at least partially to assemble the surgical theater mechanism. For example, the positioning of the surgical theater mechanism can be augmented as described in this disclosure. One embodiment of the augmenting positioning of a surgical theater mechanism at least partially to assemble the surgical theater mechanism of operation 2402 can include operation 2410 that can include, but is not limited to, augmenting positioning of a thoracic surgical theater mechanism at least partially to assemble the surgical theater mechanism. For example, the surgical theater can be applied to the thoracic wall and/or the thoracic cavity, as described in this disclosure. One embodiment of the augmenting positioning of a surgical theater mechanism at least partially to assemble the surgical theater mechanism of operation 2402 can include operation 2412 that can include, but is not limited to, augmenting positioning of an abdominal surgical theater mechanism at least partially to assemble the surgical theater mechanism. For example, the surgical theater can be applied to the abdominal wall and/or the abdominal cavity, as described in this disclosure. The order of the operations, methods, mechanisms, etc. as described with respect to FIG. 22 is intended to be illustrative in nature, and not limited in scope.

FIG. 23 shows one embodiment of the position augmenting mechanism 100 that can act to augment an originally unsecured bridging element relative to an originally unsecured attachment element. There can be variety of embodiments of the position augmenting mechanism 100 that can be configured to operate as, but is not limited to, the shunt or other fluid-flow-based device, as described with respect to FIGS. 12 and/or 13.

FIG. 24 shows one embodiment of the position augmenting mechanism 100 such as described with respect to, but not limited to, FIGS. 1 to 10, and elsewhere in this disclosure. One embodiment of a high-level flowchart of an attachment mechanism 2600 is described with respect to FIG. 24 and can include, but is not limited to, operation 2602. The high-level flowchart of FIG. 24 should be considered in combination with the embodiments of the position augmenting mechanism 100, as described with respect to FIG. 23, and elsewhere in this disclosure. One embodiment of operation 2602 can include, but is not limited to, augmenting positioning of a shunt tube with respect to a shunt device at least partially to configure the shunt device into an operable position. For example, the position of a shunt tube can be augmented relative to a body of the shunt device, as described in this disclosure with respect to FIG. 23. The order of the operations, methods, mechanisms, etc. as described with respect to FIG. 24 is intended to be illustrative in nature, and not limited in scope.

In one or more various aspects, related systems include but are not limited to circuitry and/or programming for effecting the herein-referenced method aspects; the circuitry and/or programming can be virtually any combination of hardware, software, electro-mechanical system, and/or firmware configurable to effect the herein-referenced method aspects depending upon the design choices of the system designer.

### 4. Conclusion

This disclosure provides a number of embodiments of the position augmenting mechanism. The embodiments of the position augmenting mechanism as described with respect to this disclosure are intended to be illustrative in nature, and are not limiting its scope.

Those having skill in the art will recognize that the state of the art in computer, controller, communications, networking, and other similar technologies has progressed to the point where there is little distinction left between hardware, firmware, and/or software implementations of aspects of systems, such as may be utilized in the position augmenting mechanism. The use of hardware, firmware, and/or software can therefore generally represent (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle can vary with the context in which the processes and/or systems and/or other technologies are deployed. For example, if an implementer determines that speed and accuracy are paramount, the implementer and/or designer of the position augmenting mechanism may opt for mainly a hardware and/or firmware vehicle. In alternate embodiments, if flexibility is paramount, the implementer and/or designer may opt for mainly a software implementation. In yet other embodiments, the implementer and/or designer may opt for some combination of hardware, software, and/or firmware. Hence, there are several possible techniques by which the processes and/or devices and/or other technologies described herein may be effected, none of which is inherently superior to the other in that any vehicle to be utilized is a choice dependent upon the context in which the vehicle can be deployed and the specific concerns (e.g., speed, flexibility, or predictability) of the implementer, any of which may vary.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

It is to be understood by those skilled in the art that, in general, that the terms used in the disclosure, including the drawings and the appended claims (and especially as used in the bodies of the appended claims), are generally intended as “open” terms. For example, the term “including” should be interpreted as “including but not limited to”; the term “having” should be interpreted as “having at least”; and the term “includes” should be interpreted as “includes, but is not limited to”; etc. In this disclosure and the appended claims, the terms “a”, “the”, and “at least one” positioned prior to one or more goods, items, and/or services are intended to apply inclusively to either one or a plurality of those goods, items, and/or services.

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that could have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that could have A alone, B alone, C alone, A and B together, A and C together, B and C together, and/or A, B, and C together, etc.).

Those skilled in the art will appreciate that the herein-described specific exemplary processes and/or devices and/or technologies are representative of more general processes and/or devices and/or technologies taught elsewhere herein, such as in the claims filed herewith and/or elsewhere in the present application.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method, comprising:
augmenting positioning of an originally unsecured attachment element with respect to an originally unsecured bridging element.

2. The method of claim 1, wherein the augmenting positioning of an originally unsecured attachment element with respect to an originally unsecured bridging element comprises:
improving a relative positioning of the originally unsecured attachment element with respect to the originally unsecured bridging element when the former is not in a correct range of desired position(s).

3. The method of claim 1, wherein the augmenting positioning of an originally unsecured attachment element with respect to an originally unsecured bridging element comprises:
substantially maintaining a relative positioning of the originally unsecured attachment element with respect to the originally unsecured bridging element when the former is in a correct range of desired position(s).

4. The method of claim 1, wherein the augmenting positioning of an originally unsecured attachment element with respect to an originally unsecured bridging element comprises:
augmenting positioning along three orthogonal axes of the originally unsecured attachment element relative to the originally unsecured bridging element.

5. The method of claim 1, wherein the augmenting positioning of an originally unsecured attachment element with respect to an originally unsecured bridging element comprises:
augmenting positioning along two orthogonal axes with some permissible motion along a third orthogonal axis of the originally unsecured attachment element relative to the originally unsecured bridging element.

6. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using an offset-based position augmenting mechanism.

7. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using a biased-based position augmenting mechanism.

8. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using a processor-based position augmenting mechanism.

9. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using a deformation-based position augmenting mechanism.

10. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using an inflatable-based position augmenting mechanism.

11. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using a shape memory-based position augmenting mechanism.

12. The method of claim 1, wherein the augmenting positioning of an originally unsecured attachment element with respect to an originally unsecured bridging element comprises:
augmenting positioning of the originally unsecured attachment element relative to the originally unsecured bridging element at least partially using a fastener cup attachment mechanism.

13. The method of claim 1, wherein the augmenting the originally unsecured attachment element relative to the originally unsecured bridging element can be performed substantially within an individual.

14. A method, comprising:
controlling positioning of a bony element at least partially by augmenting positioning of an originally unsecured attachment element relative to an originally unsecured bridging element.

15. The method of claim 14, wherein the controlling positioning of a bony element at least partially by augmenting positioning of an originally unsecured attachment element relative to an originally unsecured bridging element comprises:
improving a relative positioning of the originally unsecured attachment element with respect to the originally unsecured bridging element when the former is not in a correct range of desired position(s).

16. The method of claim 14, wherein the controlling positioning of a bony element at least partially by augmenting positioning of an originally unsecured attachment element relative to an originally unsecured bridging element comprises:
substantially maintaining a relative positioning of the originally unsecured attachment element with respect to the originally unsecured bridging element when the former is in a correct range of desired position(s).

17. The method of claim 14, further comprising maintaining positioning of the originally unsecured attachment element relative to the originally unsecured bridging element.

18. A method, comprising:
augmenting positioning of a surgical theater mechanism at least partially to assemble the surgical theater mechanism.

19. The method of claim 18, wherein the augmenting positioning of a surgical theater mechanism at least partially to assemble the surgical theater mechanism comprises:
augmenting positioning of a thoracic surgical theater mechanism at least partially to assemble the surgical theater mechanism.

20. The method of claim 18, wherein the augmenting positioning of a surgical theater mechanism at least partially to assemble the surgical theater mechanism comprises:
augmenting positioning of an abdominal surgical theater mechanism at least partially to assemble the surgical theater mechanism.

21. A method, comprising:
augmenting positioning of a shunt tube with respect to a shunt device at least partially to configure the shunt device into an operable position.

22. An apparatus, comprising:
A position augmenting mechanism that can augment securing an originally unsecured attachment element relative to an originally unsecured bridging element.

23. The apparatus of claim 22, wherein the position augmenting mechanism that can augment securing an originally unsecured attachment element relative to an originally unsecured bridging element comprises:
the position augmenting mechanism that can augment securing along three orthogonal axes the originally unsecured attachment element relative to the originally unsecured bridging element.

24. The apparatus of claim 22, wherein the position augmenting mechanism that can augment securing an originally unsecured attachment element relative to an originally unsecured bridging element comprises:
the position augmenting mechanism that can augment securing along two orthogonal axes with some permissible motion along a third orthogonal axis the originally unsecured attachment element relative to the originally unsecured bridging element.

25. The apparatus of claim 22, wherein the position augmenting mechanism that can augment securing an originally unsecured attachment element relative to an originally unsecured bridging element comprises:
an offset-based position augmenting mechanism that can augment the securing the originally unsecured attachment element relative to the originally unsecured bridging element.

26. The apparatus of claim 22, wherein the position augmenting mechanism that can augment securing an originally unsecured attachment element relative to an originally unsecured bridging element comprises:
a biased-based position augmenting mechanism that can augment the securing the originally unsecured attachment element relative to the originally unsecured bridging element.

27. The apparatus of claim 22, wherein the position augmenting mechanism that can augment securing an originally unsecured attachment element relative to an originally unsecured bridging element comprises:
a processor-based position augmenting mechanism that can augment the securing the originally unsecured attachment element relative to the originally unsecured bridging element.

28. The apparatus of claim 22, wherein the position augmenting mechanism that can augment securing an originally unsecured attachment element relative to an originally unsecured bridging element comprises:
a deformation-based position augmenting mechanism that can augment the securing the originally unsecured attachment element relative to the originally unsecured bridging element.

29. The apparatus of claim 22, wherein the position augmenting mechanism that can augment securing an originally unsecured attachment element relative to an originally unsecured bridging element comprises:
an inflatable-based position augmenting mechanism that can augment the securing the originally unsecured attachment element relative to the originally unsecured bridging element.

30. The apparatus of claim 22, wherein the position augmenting mechanism that can augment securing an originally unsecured attachment element relative to an originally unsecured bridging element comprises:
a pre-deformation-based position augmenting mechanism that can augment the securing the originally unsecured attachment element relative to the originally unsecured bridging element.

31. The apparatus of claim 22, wherein the position augmenting mechanism that can augment securing an originally unsecured attachment element relative to an originally unsecured bridging element comprises:
a fastener cup based position augmenting mechanism that can augment the securing the originally unsecured attachment element relative to the originally unsecured bridging element.

32. An apparatus, comprising:
a position augmenting mechanism that can be configurable to secure a bony element at least partially based on a securing an originally unsecured attachment element relative to an originally unsecured bridging element.

33. The apparatus of claim 32, wherein the position augmenting mechanism that can be configurable to secure a bony element at least partially based on a securing an originally unsecured attachment element relative to an originally unsecured bridging element comprises:
the position augmenting mechanism that can be configurable to substantially secure along three orthogonal axes the originally unsecured attachment element to the originally unsecured bridging element.

34. The apparatus of claim 32, wherein the position augmenting mechanism that can be configurable to secure a bony element at least partially based on a securing an originally unsecured attachment element relative to an originally unsecured bridging element comprises:
the position augmenting mechanism that can be configurable to substantially secure along two orthogonal axes while allowing a limited motion for the bony element along a third orthogonal axis the originally unsecured attachment element relative to the originally unsecured bridging element.

35. An apparatus, comprising:
a surgical theater mechanism that can be at least partially fabricated at least partially based on position augmenting a first surgical theater element relative to a second surgical theater element.

36. The apparatus of claim 35, wherein the surgical theater mechanism that can be at least partially fabricated at least partially based on position augmenting a first surgical theater element relative to a second surgical theater element comprises:
a thoracic surgical theater mechanism that can be at least partially fabricated at least partially based on position augmenting the first surgical theater element relative to the second surgical theater element.

37. The apparatus of claim 35, wherein the surgical theater mechanism that can be at least partially fabricated at least partially based on position augmenting a first surgical theater element relative to a second surgical theater element comprises:
an abdominal surgical theater mechanism that can be at least partially fabricated at least partially based on position augmenting the first surgical theater element relative to the second surgical theater element.

38. An apparatus, comprising:
an individual-internal position augmenting mechanism that can be attached at least partially based on securing an originally unsecured bridging element relative to an originally unsecured attachment element within a desired range of positions at least partially internally to an individual.

39. The apparatus of claim 38, wherein the individual-internal position augmenting mechanism can be applied to within the individual using minimally invasive surgical techniques.

40. The apparatus of claim 38, wherein the individual-internal position augmenting mechanism can be expanded within the individual using minimally invasive surgical techniques.

41. The apparatus of claim 38, wherein the individual-internal position augmenting mechanism can be applied to within the individual using open surgical techniques.

42. The apparatus of claim 38, wherein the individual-internal position augmenting mechanism can be expanded within the individual using open surgical techniques.

43. The apparatus of claim 38, further comprising:
the individual-internal position augmenting mechanism configurable to be adjusted within the individual.

44. The apparatus of claim 38, further comprising:
the individual-internal position augmenting mechanism configurable to be adjusted within the individual following a surgery performed at least partially within the individual.

45. The apparatus of claim 38, further comprising:
the individual-internal position augmenting mechanism configurable to be adjusted within the individual following a surgery within the individual based at least in part on input from input being provided outside of the individual.

46. The apparatus of claim 38, further comprising:
the individual-internal position augmenting mechanism configurable to maintain the originally unsecured bridging element relative to the originally unsecured attachment element.

47. A method, comprising:
a shunt tube configured to be augmented secured with respect to a shunt device, at least partially to configure the shunt device into an operable position.
